SEQUENCE LISTING (167 sequences)

SEQ ID NO: 1 - protein, 18 aa, Artificial Sequence
  peptide
STLRMELMGCDLNSCSMP

SEQ ID NO: 2 - protein, 16 aa, Artificial Sequence
  peptide
IALRMEVLGCEAQDLY

SEQ ID NO: 3 - protein, 20 aa, Artificial Sequence
  peptide
QYLNNGPQRIGRKYKKVRFM

SEQ ID NO: 4 - protein, 14 aa, Artificial Sequence
  peptide
LYGEVGDTLLIIFK

SEQ ID NO: 5 - protein, 16 aa, Artificial Sequence
  peptide
NGPQRIGRKYKKVRFM

SEQ ID NO: 6 - protein, 15 aa, Artificial Sequence
  peptide
KSQYLNNGPQRIGRK

SEQ ID NO: 7 - protein, 16 aa, Artificial Sequence
  peptide
PHGITDVRPLYSRRLP

SEQ ID NO: 8 - protein, 10 aa, Artificial Sequence
  peptide
THYSIRSTLR

SEQ ID NO: 9 - protein, 15 aa, Artificial Sequence
  peptide
KARLHLQGRSNAWRP

SEQ ID NO: 10 - protein, 10 aa, Artificial Sequence
  peptide
QDGHQWTLFF

SEQ ID NO: 11 - protein, 15 aa, Artificial Sequence
  peptide
NSLDPPLLTRYLRIH

SEQ ID NO: 12 - protein, 13 aa, Artificial Sequence
  peptide
IHPQSWVHQIALR

SEQ ID NO: 13 - protein, 13 aa, Artificial Sequence
  peptide
SSSQDGHQWTLFF

SEQ ID NO: 14 - protein, 9 aa, Artificial Sequence
  peptide
MGCDLNSCS

SEQ ID NO: 15 - protein, 19 aa, Artificial Sequence
  peptide
VHQIALRMEVLGCEAQDLY

SEQ ID NO: 16 - protein, 22 aa, Artificial Sequence
  peptide
KSQYLNNGPQRIGRKYKKVRFM

SEQ ID NO: 17 - protein, 10 aa, Artificial Sequence
  peptide
ELMGCDLNSC

SEQ ID NO: 18 - protein, 7 aa, Artificial Sequence
  peptide
GCDLNSC

SEQ ID NO: 19 - protein, 7 aa, Artificial Sequence
  peptide
ELMGCDL

SEQ ID NO: 20 - protein, 7 aa, Artificial Sequence
  peptide
CDLNSCS

SEQ ID NO: 21 - protein, 7 aa, Artificial Sequence
  peptide
MELMGCD

SEQ ID NO: 22 - protein, 10 aa, Artificial Sequence
  peptide
STLRMELMGC

SEQ ID NO: 23 - protein, 10 aa, Artificial Sequence
  peptide
GCDLNSCSMP

SEQ ID NO: 24 - protein, 7 aa, Artificial Sequence
  peptide
LNSCSMP

SEQ ID NO: 25 - protein, 10 aa, Artificial Sequence
  peptide
RMELMGCDLN

SEQ ID NO: 26 - protein, 7 aa, Artificial Sequence
  peptide
RMELMGC

SEQ ID NO: 27 - protein, 13 aa, Artificial Sequence
  peptide
LRMELMGCDLNSC

SEQ ID NO: 28 - protein, 7 aa, Artificial Sequence
  peptide
LMGCDLN

SEQ ID NO: 29 - protein, 13 aa, Artificial Sequence
  peptide
ELMGCDLNSCSMP

SEQ ID NO: 30 - protein, 7 aa, Artificial Sequence
  peptide
CEAQDLY

SEQ ID NO: 31 - protein, 10 aa, Artificial Sequence
  peptide
VLGCEAQDLY

SEQ ID NO: 32 - protein, 13 aa, Artificial Sequence
  peptide
RMEVLGCEAQDLY

SEQ ID NO: 33 - protein, 7 aa, Artificial Sequence
  peptide
MEVLGCE

SEQ ID NO: 34 - protein, 13 aa, Artificial Sequence
  peptide
MEVLGCEAQDLYG

SEQ ID NO: 35 - protein, 13 aa, Artificial Sequence
  peptide
EVLGCEAQDLYGS

SEQ ID NO: 36 - protein, 7 aa, Artificial Sequence
  peptide
RMEVLGC

SEQ ID NO: 37 - protein, 7 aa, Artificial Sequence
  peptide
EVLGCEA

SEQ ID NO: 38 - protein, 10 aa, Artificial Sequence
  peptide
IALRMEVLGC

SEQ ID NO: 39 - protein, 13 aa, Artificial Sequence
  peptide
GPQRIGRKYKKVR

SEQ ID NO: 40 - protein, 13 aa, Artificial Sequence
  peptide
PQRIGRKYKKVRF

SEQ ID NO: 41 - protein, 10 aa, Artificial Sequence
  peptide
RIGRKYKKVR

SEQ ID NO: 42 - protein, 13 aa, Artificial Sequence
  peptide
NNGPQRIGRKYKK

SEQ ID NO: 43 - protein, 10 aa, Artificial Sequence
  peptide
QRIGRKYKKV

SEQ ID NO: 44 - protein, 13 aa, Artificial Sequence
  peptide
NGPQRIGRKYKKV

SEQ ID NO: 45 - protein, 13 aa, Artificial Sequence
  peptide
QRIGRKYKKVRFM

SEQ ID NO: 46 - protein, 10 aa, Artificial Sequence
  peptide
PQRIGRKYKK

SEQ ID NO: 47 - protein, 13 aa, Artificial Sequence
  peptide
LNNGPQRIGRKYK

SEQ ID NO: 48 - protein, 10 aa, Artificial Sequence
  peptide
IGRKYKKVRF

SEQ ID NO: 49 - protein, 10 aa, Artificial Sequence
  peptide
GPQRIGRKYK

SEQ ID NO: 50 - protein, 7 aa, Artificial Sequence
  peptide
RKYKKVR

SEQ ID NO: 51 - protein, 13 aa, Artificial Sequence
  peptide
QYLNNGPQRIGRK

SEQ ID NO: 52 - protein, 10 aa, Artificial Sequence
  peptide
EVGDTLLIIF

SEQ ID NO: 53 - protein, 13 aa, Artificial Sequence
  peptide
LYGEVGDTLLIIF

SEQ ID NO: 54 - protein, 7 aa, Artificial Sequence
  peptide
DTLLIIF

SEQ ID NO: 55 - protein, 10 aa, Artificial Sequence
  peptide
VGDTLLIIFK

SEQ ID NO: 56 - protein, 10 aa, Artificial Sequence
  peptide
GEVGDTLLII

SEQ ID NO: 57 - protein, 7 aa, Artificial Sequence
  peptide
TLLIIFK

SEQ ID NO: 58 - protein, 13 aa, Artificial Sequence
  peptide
GPQRIGRKYKKVR

SEQ ID NO: 59 - protein, 13 aa, Artificial Sequence
  peptide
NGPQRIGRKYKKV

SEQ ID NO: 60 - protein, 13 aa, Artificial Sequence
  peptide
PQRIGRKYKKVRF

SEQ ID NO: 61 - protein, 10 aa, Artificial Sequence
  peptide
RIGRKYKKVR

SEQ ID NO: 62 - protein, 10 aa, Artificial Sequence
  peptide
QRIGRKYKKV

SEQ ID NO: 63 - protein, 13 aa, Artificial Sequence
  peptide
QRIGRKYKKVRFM

SEQ ID NO: 64 - protein, 7 aa, Artificial Sequence
  peptide
RKYKKVR

SEQ ID NO: 65 - protein, 10 aa, Artificial Sequence
  peptide
PQRIGRKYKK

SEQ ID NO: 66 - protein, 7 aa, Artificial Sequence
  peptide
GPQRIGR

SEQ ID NO: 67 - protein, 10 aa, Artificial Sequence
  peptide
GPQRIGRKYK

SEQ ID NO: 68 - protein, 7 aa, Artificial Sequence
  peptide
PQRIGRK

SEQ ID NO: 69 - protein, 13 aa, Artificial Sequence
  peptide
KSQYLNNGPQRIG

SEQ ID NO: 70 - protein, 13 aa, Artificial Sequence
  peptide
SQYLNNGPQRIGR

SEQ ID NO: 71 - protein, 13 aa, Artificial Sequence
  peptide
QYLNNGPQRIGRK

SEQ ID NO: 72 - protein, 10 aa, Artificial Sequence
  peptide
LNNGPQRIGR

SEQ ID NO: 73 - protein, 10 aa, Artificial Sequence
  peptide
YLNNGPQRIG

SEQ ID NO: 74 - protein, 10 aa, Artificial Sequence
  peptide
NNGPQRIGRK

SEQ ID NO: 75 - protein, 7 aa, Artificial Sequence
  peptide
DGPTKSD

SEQ ID NO: 76 - protein, 13 aa, Artificial Sequence
  peptide
HGITDVRPLYSRR

SEQ ID NO: 77 - protein, 13 aa, Artificial Sequence
  peptide
PHGITDVRPLYSR

SEQ ID NO: 78 - protein, 13 aa, Artificial Sequence
  peptide
ITDVRPLYSRRLP

SEQ ID NO: 79 - protein, 13 aa, Artificial Sequence
  peptide
GITDVRPLYSRRL

SEQ ID NO: 80 - protein, 10 aa, Artificial Sequence
  peptide
TDVRPLYSRR

SEQ ID NO: 81 - protein, 7 aa, Artificial Sequence
  peptide
RPLYSRR

SEQ ID NO: 82 - protein, 10 aa, Artificial Sequence
  peptide
ITDVRPLYSR

SEQ ID NO: 83 - protein, 10 aa, Artificial Sequence
  peptide
DVRPLYSRRL

SEQ ID NO: 84 - protein, 10 aa, Artificial Sequence
  peptide
VRPLYSRRLP

SEQ ID NO: 85 - protein, 7 aa, Artificial Sequence
  peptide
VRPLYSR

SEQ ID NO: 86 - protein, 6 aa, Artificial Sequence
  peptide
PRCLTR

SEQ ID NO: 87 - protein, 10 aa, Artificial Sequence
  peptide
THYSIRSTLR

SEQ ID NO: 88 - protein, 13 aa, Artificial Sequence
  peptide
GSGTHYSIRSTLR

SEQ ID NO: 89 - protein, 13 aa, Artificial Sequence
  peptide
ARLHLQGRSNAWR

SEQ ID NO: 90 - protein, 10 aa, Artificial Sequence
  peptide
HLQGRSNAWR

SEQ ID NO: 91 - protein, 13 aa, Artificial Sequence
  peptide
KARLHLQGRSNAW

SEQ ID NO: 92 - protein, 13 aa, Artificial Sequence
  peptide
RLHLQGRSNAWRP

SEQ ID NO: 93 - protein, 10 aa, Artificial Sequence
  peptide
LHLQGRSNAW

SEQ ID NO: 94 - protein, 7 aa, Artificial Sequence
  peptide
GRSNAWR

SEQ ID NO: 95 - protein, 10 aa, Artificial Sequence
  peptide
QDGHQWTLFF

SEQ ID NO: 96 - protein, 7 aa, Artificial Sequence
  peptide
GHQWTLF

SEQ ID NO: 97 - protein, 7 aa, Artificial Sequence
  peptide
HQWTLFF

SEQ ID NO: 98 - protein, 7 aa, Artificial Sequence
  peptide
LLTRYLR

SEQ ID NO: 99 - protein, 7 aa, Artificial Sequence
  peptide
LTRYLRI

SEQ ID NO: 100 - protein, 7 aa, Artificial Sequence
  peptide
PLLTRYL

SEQ ID NO: 101 - protein, 7 aa, Artificial Sequence
  peptide
TRYLRIH

SEQ ID NO: 102 - protein, 13 aa, Artificial Sequence
  peptide
NSLDPPLLTRYLR

SEQ ID NO: 103 - protein, 10 aa, Artificial Sequence
  peptide
DPPLLTRYLR

SEQ ID NO: 104 - protein, 10 aa, Artificial Sequence
  peptide
PPLLTRYLRI

SEQ ID NO: 105 - protein, 13 aa, Artificial Sequence
  peptide
IHPQSWVHQIALR

SEQ ID NO: 106 - protein, 7 aa, Artificial Sequence
  peptide
VHQIALR

SEQ ID NO: 107 - protein, 7 aa, Artificial Sequence
  peptide
HQWTLFF

SEQ ID NO: 108 - protein, 10 aa, Artificial Sequence
  peptide
QDGHQWTLFF

SEQ ID NO: 109 - protein, 13 aa, Artificial Sequence
  peptide
SSSQDGHQWTLFF

SEQ ID NO: 110 - protein, 10 aa, Artificial Sequence
  peptide
SQDGHQWTLF

SEQ ID NO: 111 - protein, 7 aa, Artificial Sequence
  peptide
SQDGHQW

SEQ ID NO: 112 - protein, 7 aa, Artificial Sequence
  peptide
GHQWTLF

SEQ ID NO: 113 - protein, 7 aa, Artificial Sequence
  peptide
GCDLNSC

SEQ ID NO: 114 - protein, 10 aa, Artificial Sequence
  peptide
LRMELMGCDL

SEQ ID NO: 115 - protein, 10 aa, Artificial Sequence
  peptide
ELMGCDLNSC

SEQ ID NO: 116 - protein, 10 aa, Artificial Sequence
  peptide
STLRMELMGC

SEQ ID NO: 117 - protein, 10 aa, Artificial Sequence
  peptide
LMGCDLNSCS

SEQ ID NO: 118 - protein, 7 aa, Artificial Sequence
  peptide
ELMGCDL

SEQ ID NO: 119 - protein, 10 aa, Artificial Sequence
  peptide
TLRMELMGCD

SEQ ID NO: 120 - protein, 13 aa, Artificial Sequence
  peptide
LRMELMGCDLNSC

SEQ ID NO: 121 - protein, 7 aa, Artificial Sequence
  peptide
RMEVLGC

SEQ ID NO: 122 - protein, 10 aa, Artificial Sequence
  peptide
IALRMEVLGC

SEQ ID NO: 123 - protein, 10 aa, Artificial Sequence
  peptide
ALRMEVLGCE

SEQ ID NO: 124 - protein, 13 aa, Artificial Sequence
  peptide
VHQIALRMEVLGC

SEQ ID NO: 125 - protein, 10 aa, Artificial Sequence
  peptide
LGCEAQDLYG

SEQ ID NO: 126 - protein, 7 aa, Artificial Sequence
  peptide
EVLGCEA

SEQ ID NO: 127 - protein, 15 aa, Artificial Sequence
  peptide
CGRPQKRPSCIGCKG

SEQ ID NO: 128 - protein, 22 aa, Artificial Sequence
  peptide
LKWNPDDYGGVKKIHIPSEKGC

SEQ ID NO: 129 - protein, 13 aa, Artificial Sequence
  peptide
DHTLYTPYHTHPG

SEQ ID NO: 130 - protein, 11 aa, Artificial Sequence
  peptide
GRPQKRPSCIG

SEQ ID NO: 131 - protein, 11 aa, Artificial Sequence
  peptide
VKKIHIPSEKG

SEQ ID NO: 132 - protein, 12 aa, Artificial Sequence
  peptide
GGVKKIHIPSEK

SEQ ID NO: 133 - protein, 12 aa, Artificial Sequence
  peptide
VKKIHIPSEKGC

SEQ ID NO: 134 - protein, 10 aa, Artificial Sequence
  peptide
IPNPLLGLDC

SEQ ID NO: 135 - protein, 23 aa, Artificial Sequence
  peptide
IPNPLLGLDGGSGDYKDDDDKGK

SEQ ID NO: 136 - protein, 24 aa, Artificial Sequence
  peptide
VKKIHIPSEKGGSGDYKDDDDKGK

SEQ ID NO: 137 - protein, 116 aa, Artificial Sequence
  heavy chain variable (VH) region
DVQLQESGPGLVKPSQSLSLTCTVTGYSITSDYAWNWIRQFPGNKLEWMGYITYSGITNYNPSLKSQISITRDTSKNQFFLQLNSVTTEDTATYYCVSGTYYFDYWGQGTTLTVSS

SEQ ID NO: 138 - protein, 109 aa, Artificial Sequence
  light-chain variable (VL) region
SVVMTQTPKSLLISIGDRVTITCKASQSVSSDVAWFQQKPGQSPKPLIYYASNRYTGVPDRFTGSGYGTDFTFTISSVQAEDLAVYFCGQDYTSPRTFGGGTKLEIKRA

SEQ ID NO: 139 - protein, 25 aa, Artificial Sequence
  peptide
CSGRVEVKVQEEWGTVCNNGWSMEA

SEQ ID NO: 140 - protein, 11 aa, Artificial Sequence
  peptide
GRVEVKVQEEW

SEQ ID NO: 141 - protein, 10 aa, Artificial Sequence
  peptide
WGTVCNNGWS

SEQ ID NO: 142 - protein, 9 aa, Artificial Sequence
  peptide
WGTVCNNGW

SEQ ID NO: 143 - protein, 13 aa, Artificial Sequence
  peptide
EWGTVCNNGWSME

SEQ ID NO: 144 - protein, 13 aa, Artificial Sequence
  peptide
QEEWGTVCNNGWS

SEQ ID NO: 145 - protein, 13 aa, Artificial Sequence
  peptide
WGTVCNNGWSMEA

SEQ ID NO: 146 - protein, 13 aa, Artificial Sequence
  peptide
EEWGTVCNNGWSM

SEQ ID NO: 147 - protein, 13 aa, Artificial Sequence
  peptide
VQEEWGTVCNNGW

SEQ ID NO: 148 - protein, 10 aa, Artificial Sequence
  peptide
EWGTVCNNGW

SEQ ID NO: 149 - protein, 21 aa, Artificial Sequence
  peptide
DHVSCRGNESALWDCKHDGWG

SEQ ID NO: 150 - protein, 5 aa, Artificial Sequence
  peptide
ESALW

SEQ ID NO: 151 - protein, 7 aa, Artificial Sequence
  peptide
ESALWDC

SEQ ID NO: 152 - protein, 10 aa, Artificial Sequence
  peptide
RGNESALWDC

SEQ ID NO: 153 - protein, 10 aa, Artificial Sequence
  peptide
SCRGNESALW

SEQ ID NO: 154 - protein, 13 aa, Artificial Sequence
  peptide
VSCRGNESALWDC

SEQ ID NO: 155 - protein, 10 aa, Artificial Sequence
  peptide
ALWDCKHDGW

SEQ ID NO: 156 - protein, 13 aa, Artificial Sequence
  peptide
DHVSCRGNESALW

SEQ ID NO: 157 - protein, 10 aa, Artificial Sequence
  peptide
CRGNESALWD

SEQ ID NO: 158 - protein, 13 aa, Artificial Sequence
  peptide
NESALWDCKHDGW

SEQ ID NO: 159 - protein, 13 aa, Artificial Sequence
  peptide
ESALWDCKHDGWG

SEQ ID NO: 160 - protein, 20 aa, Artificial Sequence
  peptide
SSLGGTDKELRLVDGENKCS

SEQ ID NO: 161 - protein, 11 aa, Artificial Sequence
  peptide
SSLGGTDKELR

SEQ ID NO: 162 - protein, 5 aa, Artificial Sequence
  peptide
SSLGG

SEQ ID NO: 163 - protein, 10 aa, Artificial Sequence
  peptide
SSLGGTDKEL

SEQ ID NO: 164 - protein, 9 aa, Artificial Sequence
  peptide
SSLGGTDKE

SEQ ID NO: 165 - protein, 8 aa, Artificial Sequence
  peptide
SSLGGTDK

SEQ ID NO: 166 - protein, 7 aa, Artificial Sequence
  peptide
SSLGGTD

SEQ ID NO: 167 - protein, 6 aa, Artificial Sequence
  peptide
SSLGGT

(19) United States
(12) Patent Application Publication
(10) Pub. No.: US 2023/0365655 A1
(43) Pub. Date: Nov. 16, 2023

(54) COMPOUND FOR INCREASING THE EFFICACY OF FACTOR VIII REPLACEMENT THERAPY

(71) Applicant: ABLEVIA BIOTECH GMBH, Wien (AT)

(72) Inventors: Oskar SMRZKA, Wien (AT); Bettina WANKO, Wien (AT)

(21) Appl. No.: 18/245,991

(22) PCT Filed: Sep. 23, 2021
(86) PCT No.: PCT/EP2021/076182

§ 371 Date: Mar. 20, 2023

(30) Foreign Application Priority Data
Sep. 23, 2020 (EP) 20197701.4

(51) Int. Cl. C07K 14/755; A61K 47/64; A61P 7/04

(52) U.S. Cl. CPC C07K 14/755; A61K 47/644; A61K 47/643; A61P 7/04

(57) ABSTRACT

A compound for the sequestration of undesirable antibodies associated with hemophilia A, in particular when treated by factor VIII replacement therapy. The compound includes a biopolymer scaffold and at least two peptides derived from factor VIII with a sequence length of 6-13 amino acid, wherein each of the peptides independently includes a 6-amino-acid fragment of the amino-acid sequence of factor VIII, optionally wherein at most three, preferably at most two, more preferably at most one amino acid is independently substituted by any other amino acid. Also provided are pharmaceutical compositions including the compound, as well as methods for treating hemophilia A.

The field of present invention relates to the therapy of hemophilia A.

Hemophilia A is typically described as a disease in which blood clotting is impaired because of a deficiency or inhibition of the clotting factor VIII (Peters & Harris, 2018). As a consequence of impaired blood clotting, excessive bleeding can occur either spontaneously or secondary to trauma because the blood from wounds does not clot or clots only slowly. Spontaneous bleeding can occur in episodes and one of the most common manifestations of hemophilia A can be subcutaneous and muscle bleeding, but also gastrointestinal-, genitourinary- and retroperitoneal bleeding. Hemophilia patients may also suffer from bleeding into joints which can cause hemophilic arthropathy. Intracranial bleeding is rare, but can become life-threatening (Ljung 2007).

A common cause for factor VIII deficiencies is an X-linked gene defect that leads to factor VIII absence or decrease (Konkle et al 2000 [updated 2017]). Hemophilia A is considered the most common hereditary disorder of hemostasis and it occurs in 1/5000 males. Females are carriers of these mutations, and they normally do not manifest hemophilia, but occasionally have reduced factor VIII.

In contrast to the classical hereditary factor VIII deficiency (often simply referred to as “hemophilia A”), acquired hemophilia is an autoimmune disease where inhibitory autoantibodies against clotting factors (most commonly factor VIII, in which case the disease is referred to as acquired hemophilia A) are produced by the body (Franchini et al. 2017). Acquired hemophilia is typically neither associated with hereditary hemophilia nor with a family history of hemorrhages.

As the therapy of choice, factor VIII (FVIII) replacement therapy is still the standard treatment for hemophilia A (both congenital and acquired) patients. Normally factor VIII (FVIII) replacement is effective unless a patient develops factor VIII-inhibitory or -neutralizing antibodies (inhibitors) against the exogenously applied factor VIII (O'Mahony, 2020; Pratt et al, 2020). The development of neutralizing antibodies by the patient is considered the most significant treatment complication in hemophilia A and they occur in a large portion of hemophilia patients. Neutralizing antibodies can be a major burden causing considerable morbidity and a decreased quality of life. This complication often requires increased dosage, desensitization or even immunosuppression (Giangrande et al, 2018).

Lavigne-Lissalde et al. relates to anti-FVIII antibodies and discusses various approaches to address the problem of anti-FVIIII antibodies in hemophilia patients.

Ananyeva et al. reviews mechanisms of inhibition, management and perspectives in respect to inhibitors of hemophilia A. Specifically, peptide decoys for blocking FVIII inhibitors, bypassing them with human/porcine FVIII hybrids, neutralizing FVIII-reactive CD4+ T cells with anti-clonotypic antibodies, and inducing immune tolerance to FVIII with the use of universal CD4+ epitopes are discussed.

Lacroix-Desmazes et al. describes interventions, mostly in the pre-clinical stage, to prevent or reverse FVIII inhibitor development in hemophilia patients. Disclosed tolerogenic therapies include development of FVIII-Fc fusion proteins, nanoparticle-based therapies, oral tolerance, and engineering of regulatory or cytotoxic T cells to render them FVIII-specific.

Villard et al. concerns peptide decoys selected by phage display to block in vitro and in vivo activity of a human anti-FVIII inhibitor. These peptides (found in a phage display library screen with human anti-FVIII monoclonal antibody BO2C11 as a prototypical FVIII inhibitor) are disclosed to neutralize the inhibitory activity of BO2C11 in vitro and in vivo (when BO2C11 is pre-incubated with such a peptide before administration of BO2C11 to a murine model of hemophilia).

WO 2014/072958 A1 discloses peptides derivable from FVIII which are capable of binding to an MHC class II molecule without further antigen processing and being recognised by a FVIII specific T cell. Such peptides may comprise FVIII-derived sequences that contain additional terminal K or G residues, which results in a sequence length of 21 amino acids. These peptides are disclosed to induce or restore tolerance to FVIII in haemophilia patients.

Neutralizing antibodies are also the main reason why much effort has been put into new alternatives to factor VIII replacement therapies which however have disadvantages of their own. For instance, WO 2011/060371 A2 relates to FVIII T cell epitope variants having reduced immunogenicity. Disclosed are modified FVIII polypeptides with at least one amino acid modification in the C2 domain and/or the A2 domain. Recent advances in gene therapy are not expected to solve the problem of Factor VIII inhibitors (Patel 2020).

It is an object of the present invention to provide compounds and methods to improve the efficacy and/or safety of a factor VIII replacement therapy (or to provide a new treatment option for hemophilia A).

The present invention provides a compound (typically for the sequestration, or depletion, of antibodies, in particular antibodies specific for factor VIII, present in a human individual) comprising a biopolymer scaffold and at least two peptides, preferably derived from (human) factor VIII, with a sequence length of 6-13 amino acids, wherein each of the peptides independently comprises a 6-amino-acid fragment, preferably a 7-, more preferably an 8-, even more preferably a 9-, even more preferably a 10-, even more preferably an 11-, yet even more preferably a 12-, most preferably a 13-amino-acid fragment, of the amino-acid sequence of (preferably human) factor VIII, preferably as identified by UniProt accession code P00451, optionally wherein at most three, preferably at most two, more preferably at most one amino acid is independently substituted by any other amino acid.

Furthermore, the present invention provides a pharmaceutical composition comprising the compound according to the invention and at least one pharmaceutically acceptable excipient.

In an aspect, this pharmaceutical composition is for use in prevention or treatment of hemophilia A, preferably congenital hemophilia A and/or acquired hemophilia A, in an individual, preferably a human individual.

In another aspect, the pharmaceutical composition is for use in inhibiting neutralization and/or inhibition of a factor VIII replacement product, preferably factor VIII, more preferably human factor VIII, in an individual, preferably a human individual, preferably wherein the pharmaceutical composition is administered at least twice within a 96-hour window, wherein the window is followed by administration of the factor VIII replacement product within 24 hours.

In the course of the present invention, a compound was developed which is able to deplete (or sequester) antibodies against factor VIII in vivo and is therefore suitable for use in the prevention or treatment of hemophilia A (alone, especially in the case of acquired hemophilia A, as well as in combination with factor VIII replacement products).

Further, it was surprisingly found that the approach which is also used in the invention is particularly effective in reducing titres of undesired antibodies in an individual. In particular, the compound achieved especially good results with regard to selectivity, duration of titre reduction and/or level of titre reduction in an in vivo model (see experimental examples).

The detailed description given below relates to all of the above aspects of the invention unless explicitly excluded.

In general, antibodies are essential components of the humoral immune system, offering protection from infections by foreign organisms including bacteria, viruses, fungi or parasites. However, under certain circumstances - including autoimmune diseases, organ transplantation, blood transfusion or upon administration of biomolecular drugs or gene delivery vectors—antibodies can target the patient's own body (or the foreign tissue or cells or the biomolecular drug or vector just administered), thereby turning into harmful or disease-causing entities. Certain antibodies can also interfere with probes for diagnostic imaging. In the following, such antibodies are generally referred to as “undesired antibodies” or “undesirable antibodies”.

With few exceptions, selective removal of undesired antibodies has not reached clinical practice. It is presently restricted to very few indications: One of the known techniques for selective antibody removal (although not widely established) is immunoapheresis. In contrast to immunoapheresis (which removes immunoglobulin), selective immunoapheresis involves the filtration of plasma through an extracorporeal, selective antibody-adsorber cartridge that will deplete the undesired antibody based on selective binding to its antigen binding site. Selective immunoapheresis has for instance been used for removing anti-A or anti-B antibodies from the blood prior to ABO-incompatible transplantation or with respect to indications in transfusion medicine (Teschner et al). Selective apheresis was also experimentally applied in other indications, such as neuroimmunological indications (Tetala et al) or myasthenia gravis (Lazaridis et al), but is not yet established in the clinical routine. One reason that selective immunoapheresis is only hesitantly applied is the fact that it is a cost intensive and cumbersome intervention procedure that requires specialized medical care. Moreover, it is not known in the prior art how to deplete undesired antibodies rapidly and efficiently.

Unrelated to apheresis, Morimoto et al. discloses dextran as a generally applicable multivalent scaffold for improving immunoglobulin-binding affinities of peptide and peptidomimetic ligands such as the FLAG peptide. WO 2011/130324 A1 relates to compounds for prevention of cell injury. EP 3 059 244 A1 relates to a C-met protein agonist.

As mentioned, apheresis is applied extracorporeally. By contrast, also several approaches to deplete undesirable antibodies intracorporeally were proposed in the prior art, mostly in connection with certain autoimmune diseases involving autoantibodies or anti-drug antibodies:

Lorentz et al discloses a technique whereby erythrocytes are charged in situ with a tolerogenic payload driving the deletion of antigen-specific T cells. This is supposed to ultimately lead to reduction of the undesired humoral response against a model antigen. A similar approach is proposed in Pishesha et al. In this approach, erythrocytes are loaded ex vivo with a peptide-antigen construct that is covalently bound to the surface and reinjected into the animal model for general immunotolerance induction.

WO 92/13558 A1 relates to conjugates of stable nonimmunogenic polymers and analogues of immunogens that possess the specific B cell binding ability of the immunogen and which, when introduced into individuals, induce humoral anergy to the immunogen. Accordingly, these conjugates are disclosed to be useful for treating antibody-mediated pathologies that are caused by foreign- or self-immunogens. In this connection, see also EP 0 498 658 A2.

Taddeo et al discloses selectively depleting antibody producing plasma cells using anti-CD138 antibody derivatives fused to an ovalbumin model antigen thereby inducing receptor crosslinking and cell suicide in vitro selectively in those cells that express the antibody against the model antigen.

Apitope International NV (Belgium) is presently developing soluble tolerogenic T-cell epitope peptides which may lead to expression of low levels of co-stimulatory molecules from antigen presenting cells inducing tolerance, thereby suppressing antibody response (see e.g. Jansson et al). These products are currently under preclinical and early clinical evaluation, e.g. in multiple sclerosis, Grave's disease, intermediate uveitis, and other autoimmune conditions as well as Factor VIII intolerance.

Similarly, Selecta Biosciences, Inc. (USA) is currently pursuing strategies of tolerance induction by so-called Synthetic Vaccine Particles (SVPs). SVP-Rapamycin is supposed to induce tolerance by preventing undesired antibody production via selectively inducing regulatory T cells (see Mazor et al).

Mingozzi et al discloses decoy adeno-associated virus (AAV) capsids that adsorb antibodies but cannot enter a target cell.

WO 2015/136027 Al discloses carbohydrate ligands presenting the minimal Human Natural Killer-1 (HNK-1) epitope that bind to anti-MAG (myelin-associated glycoprotein) IgM antibodies, and their use in diagnosis as well as for the treatment of anti-MAG neuropathy. WO 2017/046172 A1 discloses further carbohydrate ligands and moieties, respectively, mimicking glycoepitopes comprised by glycosphingolipids of the nervous system which are bound by anti-glycan antibodies associated with neurological diseases. The document further relates to the use of these carbohydrate ligands/moieties in diagnosis as well as for the treatment of neurological diseases associated with anti-glycan antibodies.

US 2004/0258683 A1 discloses methods for treating systemic lupus erythematosus (SLE) including renal SLE and methods of reducing risk of renal flare in individuals with SLE, and methods of monitoring such treatment. One disclosed method of treating SLE including renal SLE and reducing risk of renal flare in an individual with SLE involves the administration of an effective amount of an agent for reducing the level of anti-double-stranded DNA (dsDNA) antibody, such as a dsDNA epitope as in the form of an epitope-presenting carrier or an epitope-presenting valency platform molecule, to the individual.

U.S. Pat. No. 5,637,454 relates to assays and treatments of autoimmune diseases. Agents used for treatment might include peptides homologous to the identified antigenic, molecular mimicry sequences. It is disclosed that these peptides could be delivered to a patient in order to decrease the amount of circulating antibody with a particular specificity.

US 2007/0026396 A1 relates to peptides directed against antibodies, which cause cold-intolerance, and the use thereof. It is taught that by using the disclosed peptides, in vivo or ex vivo neutralization of undesired autoantibodies is possible. A comparable approach is disclosed in WO 1992/014150 A1 or in WO 1998/030586 A2.

WO 2018/102668 A1 discloses a fusion protein for selective degradation of disease-causing or otherwise undesired antibodies. The fusion protein (termed “Seldeg”) includes a targeting component that specifically binds to a cell surface receptor or other cell surface molecule at near-neutral pH, and an antigen component fused directly or indirectly to the targeting component. Also disclosed is a method of depleting a target antigen-specific antibody from a patient by administering to the patient a Seldeg having an antigen component configured to specifically bind the target antigen-specific antibody.

WO 2015/181393 A1 concerns peptides grafted into sunflower-trypsin-inhibitor- (SFTI-) and cyclotide-based scaffolds. These peptides are disclosed to be effective in autoimmune disease, for instance citrullinated fibrinogen sequences that are grafted into the SFTI scaffold have been shown to block autoantibodies in rheumatoid arthritis and inhibit inflammation and pain. These scaffolds are disclosed to be non-immunogenic.

Erlandsson et al discloses in vivo clearing of idiotypic antibodies with anti-idiotypic antibodies and their derivatives.

Berlin Cures Holding AG (Germany) has proposed an intravenous broad spectrum neutralizer DNA aptamer (see e.g. WO 2016/020377 A1 and WO 2012/000889 A1) for the treatment of dilated cardiomyopathy and other GPCR-autoantibody related diseases that in high dosage is supposed to block autoantibodies by competitive binding to the antigen binding regions of autoantibodies. In general, aptamers did not yet achieve a breakthrough and are still in a preliminary stage of clinical development. The major concerns are still biostability and bioavailability, constraints such as nuclease sensitivity, toxicity, small size and renal clearance. A particular problem with respect to their use as selective antibody antagonists are their propensity to stimulate the innate immune response.

WO 00/33887 A2 discloses methods for reducing circulating levels of antibodies, particularly disease-associated antibodies. The methods entail administering effective amounts of epitope-presenting carriers to an individual. In addition, ex vivo methods for reducing circulating levels of antibodies are disclosed which employ epitope-presenting carriers.

U.S. Pat. No. 6,022,544 A relates to a method for reducing an undesired antibody response in a mammal by administering to the mammal a non-immunogenic construct which is free of high molecular weight immunostimulatory molecules. The construct is disclosed to contain at least two copies of a B cell membrane immunoglobulin receptor epitope bound to a pharmaceutically acceptable non-immunogenic carrier.

However, the approaches to deplete undesirable antibodies intracorporeally disclosed in the prior art have many shortcomings. In particular, neither of them has been approved for regular clinical use.

With respect to the compound of the present invention, it is preferred that each of the peptides independently comprises a 6-, preferably a 7-, more preferably an 8-, even more preferably a 9-, even more preferably a 10-, yet even more preferably an 11-, yet even more preferably a 12-, most preferably a 13-amino-acid fragment of an amino-acid sequence selected from the group consisting of: STLRMELMGCDLNSCSMP (SEQ ID NO: 1), IALRMEVLGCEAQDLY (SEQ ID NO: 2), QYLNNGPQRIGRKYKKVRFM (SEQ ID NO: 3), LYGEVGDTLLIIFK (SEQ ID NO: 4), NGPQRIGRKYKKVRFM (SEQ ID NO: 5), KSQYLNNGPQRIGRK (SEQ ID NO: 6), PHGITDVRPLYSRRLP (SEQ ID NO: 7), THYSIRSTLR (SEQ ID NO: 8), KARLHLQGRSNAWRP (SEQ ID NO: 9), QDGHQWTLFF (SEQ ID NO: 10), NSLDPPLLTRYLRIH (SEQ ID NO: 11), IHPQSWVHQIALR (SEQ ID NO: 12), SSSQDGHQWTLFF (SEQ ID NO: 13), MGCDLNSCS (SEQ ID NO: 14), VHQIALRMEVLGCEAQDLY (SEQ ID NO: 15), and KSQYLNNGPQRIGRKYKKVRFM (SEQ ID NO: 16). Especially preferred epitopes in this context are SSSQDGHQWTLFF (SEQ ID NO: 13), VHQIALRMEVLGCEAQDLY (SEQ ID NO: 15), and KSQYLNNGPQRIGRKYKKVRFM (SEQ ID NO: 16).

Preferably, the at least two peptides comprise a peptide P₁ and a peptide P₂, wherein P₁ and P₂ independently comprise a 6-, preferably a 7-, more preferably an 8-, even more preferably a 9-, even more preferably a 10-, yet even more preferably an 11-, especially a 12-, most preferably a 13-amino-acid fragment of an amino-acid sequence selected from SEQ ID NO: 1 to 16, wherein P₁ and P₂ are present in form of a peptide dimer P₁-S -P₂, wherein S is a non-peptide spacer, wherein the peptide dimer is covalently bound to the biopolymer scaffold, preferably via a linker.

A preferred embodiment of the inventive compound relates to a compound comprising
a biopolymer scaffold and at least
a first peptide n-mer of the general formula:

P (-S-P)₍ₙ₋₁₎ and
a second peptide n-mer of the general formula:

P (-S-P)₍ₙ₋₁₎;
wherein, independently for each occurrence, P is a peptide and S is a non-peptide spacer,
wherein, independently for each of the peptide n-mers, n is an integer of at least 1, preferably of at least 2, more preferably of at least 3, especially of at least 4,
wherein each of the peptide n-mers is bound to the biopolymer scaffold, preferably via a linker each. “P” in this context is defined, independently for each occurrence, in the same way as disclosed for the at least two peptides of the inventive compound and/or as for P₁ and P₂ defined above.

According to a preferred embodiment of the inventive compound, each of the peptides (e.g. said at least two peptides, P₁, P₂, and/or P) independently comprises an amino-acid sequence selected from SEQ ID NOs: 17 to 126, optionally wherein at most three, preferably at most two, more preferably at most one amino acid is independently substituted by any other amino acid. Alternatively (or in addition), each of the peptides independently comprises a 6-, preferably a 7-, more preferably an 8-, more preferably a 9-, even more preferably a 10-yet even more preferably an 11-, most preferably a 12-amino-acid fragment of an amino-acid sequence selected from SEQ ID NOs: 17 to 126, optionally wherein at most three, preferably at most two, more preferably at most one amino acid is independently substituted by any other amino acid.

In a further preferred embodiment, each of the peptides (e.g. said at least two peptides, P₁, P₂, and/or P) independently consists an amino-acid sequence selected from SEQ ID NOs: 17 to 126, optionally wherein at most three, preferably at most two, more preferably at most one amino acid is independently substituted by any other amino acid, optionally with an N-terminal and/or C-terminal cysteine residue.

According to another preferred embodiment, the respective amino acid sequences of the at least two peptides of the inventive compound are the same. In other words, the at least two peptides are identical.

The biopolymer scaffold used in the present invention may be a mammalian biopolymer such as a human biopolymer, a non-human primate biopolymer, a sheep biopolymer, a pig biopolymer, a dog biopolymer or a rodent biopolymer. In particular the biopolymer scaffold is a protein, especially a (non-modified or non- modified with respect to its amino-acid sequence) plasma protein. Preferably, the biopolymer scaffold is a mammalian protein such as a human protein, a non-human primate protein, a sheep protein, a pig protein, a dog protein or a rodent protein. Typically, the biopolymer scaffold is a non-immunogenic and/or non-toxic protein that preferably circulates in the plasma of healthy (human) individuals and can e.g. be efficiently scavenged or recycled by scavenging receptors, such as e.g. present on myeloid cells or on liver sinusoidal endothelial cells (reviewed by Sorensen et al 2015).

According to a particular preference, the biopolymer scaffold is a (preferably human) globulin, preferably selected from the group consisting of immunoglobulins, alphal-globulins, alpha2-globulins and beta-globulins, in particular immunoglobulin G, haptoglobin and transferrin. Haptoglobin in particular has several advantageous properties, as shown in Examples 5-9, especially an advantageous safety profile.

The biopolymer scaffold may also be (preferably human) albumin, hemopexin, alpha-1-antitrypsin, C1 esterase inhibitor, lactoferrin or non-immunogenic (i.e. non-immunogenic in the individual to be treated) fragments of all of the aforementioned proteins, including the globulins.

In another preference, the biopolymer scaffold is an anti-CD163 antibody (i.e. an antibody specific for a CD163 protein) or CD163-binding fragment thereof.

Human CD163 (Cluster of Differentiation 163) is a 130 kDa membrane glycoprotein (formerly called M130) and prototypic class I scavenger receptor with an extracellular portion consisting of nine scavenger receptor cysteine-rich (SRCR) domains that are responsible for ligand binding. CD163 is an endocytic receptor present on macrophages and monocytes, it removes hemoglobin/haptoglobin complexes from the blood but it also plays a role in anti-inflammatory processes and wound healing. Highest expression levels of CD163 are found on tissue macrophages (e.g. Kupffer cells in the liver) and on certain macrophages in spleen and bone marrow. Because of its tissue-and cell-specific expression and entirely unrelated to depletion of undesirable antibodies, CD163 is regarded as a macrophage target for drug delivery of e.g. immunotoxins, liposomes or other therapeutic compound classes (Skytthe et al., 2020).

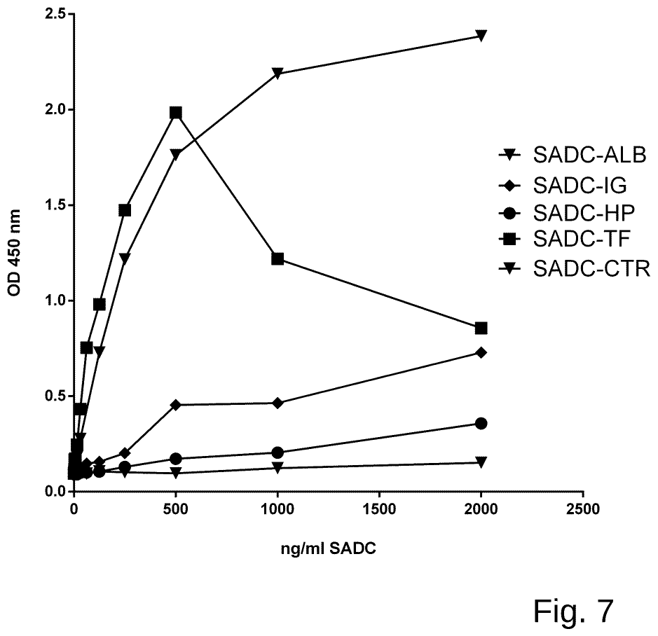

Monoclonal anti-CD163 antibodies and the SRCR domains they are binding are for instance disclosed in Madsen et al., 2004, in particular FIG. 7. Further anti-CD163 antibodies and fragments thereof are e.g. disclosed in WO 2002/032941 A2 or WO 2011/039510 A2. At least two structurally different binding sites for ligands were mapped by using domain-specific antibodies such as e.g. monoclonal antibody (mAB) EDhul (see Madsen et al, 2004). This antibody binds to the third SRCR of CD163 and competes with hemoglobin/haptoglobin binding to CD163. Numerous other antibodies against different domains of CD163 were previously described in the literature, including Mac2-158, KiM8, GHI/61 and RM3/1, targeting SRCR domains 1, 3, 7 and 9, respectively. In addition, conserved bacterial binding sites were mapped and it was demonstrated that certain antibodies were able to inhibit either bacterial binding but not hemoglobin/haptoglobin complex binding and vice versa. This points to different modes of binding and ligand interactions of CD163 (Fabriek et al, 2009; see also citations therein).

Entirely unrelated to depletion of undesirable antibodies, CD163 was proposed as a target for cell-specific drug delivery because of its physiological properties. Tumor-associated macrophages represent one of the main targets where the potential benefit of CD163-targeting is currently explored. Remarkably, numerous tumors and malignancies were shown to correlate with CD163 expression levels, supporting the use of this target for tumor therapy. Other proposed applications include CD163 targeting by anti-drug conjugates (ADCs) in chronic inflammation and neuroinflammation (reviewed in Skytthe et al., 2020). Therefore, CD163-targeting by ADCs notably with dexamethasone or stealth liposome conjugates represents therapeutic principle which is currently studied (Graversen et al., 2012; Etzerodt et al., 2012).

In that context, there are references indicating that anti-CD163 antibodies can be rapidly internalized by endocytosis when applied in vivo. This was shown for example for mAB Ed-2 (Dijkstra et al., 1985; Graversen et al., 2012) or for mAB Mac2-158/KN2/NRY (Granfeldt et al., 2013). Based on those observations in combination with observations made in the course of the present invention (see in particular example section), anti-CD163 antibodies and CD163-binding turned out to be highly suitable biopolymer scaffolds for depletion/sequestration of undesirable antibodies.

Numerous anti-CD163 antibodies and CD163-binding fragments thereof are known in the art (see e.g. above). These are suitable to be used as a biopolymer scaffold for the present invention. For instance, any anti-CD163 antibody or fragment thereof mentioned herein or in WO 2011/039510 A2 (which is included herein by reference) may be used as a biopolymer scaffold in the invention. Preferably, the biopolymer scaffold of the inventive compound is antibody Mac2-48, Mac2-158, 5C6-FAT, BerMac3, or E10B10 as disclosed in WO 2011/039510, in particular humanised Mac2-48 or Mac2-158 as disclosed in WO 2011/039510 A2.

In a preferred embodiment, the anti-CD163 antibody or CD163-binding fragment thereof comprises a heavy-chain variable (VH) region comprising one or more complementarity-determining region (CDR) sequences selected from the group consisting of SEQ ID NOs: 11-13 of WO 2011/039510 A2.

In addition, or alternatively thereto, in a preferred embodiment, the anti-CD163 antibody or CD163-binding fragment thereof comprises a light-chain variable (V_{L}) region comprising one or more CDR sequences selected from the group consisting of SEQ ID NOs: 14-16 of WO 2011/039510 A2 or selected from the group consisting of SEQ ID NOs:17-19 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 20 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 21 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 22 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 23 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 24 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 25 of WO 2011/039510 A2.

In the context of the present invention, the anti-CD163 antibody may be a mammalian antibody such as a humanized or human antibody, a non-human primate antibody, a sheep antibody, a pig antibody, a dog antibody or a rodent antibody. In embodiments, the anti-CD163 antibody may monoclonal.

According to a preference, the anti-CD163 antibody is selected from IgG, IgA, IgD, IgE and IgM.

According to a further preference, the CD163-binding fragment is selected from a Fab, a Fab′, a F(ab)2, a Fv, a single-chain antibody, a nanobody and an antigen-binding domain.

CD163 amino acid sequences are for instance disclosed in WO 2011/039510 A2 (which is included here by reference). In the context of the present invention, the anti-CD163 antibody or CD163-binding fragment thereof is preferably specific for a human CD163, especially with the amino acid sequence of any one of SEQ ID NOs: 28-31 of WO 2011/039510 A2.

In a further preferred embodiment, the anti-CD163 antibody or CD163-binding fragment thereof is specific for the extracellular region of CD163 (e.g. for human CD163: amino acids 42-1050 of UniProt Q86VB7, sequence version 2), preferably for an SRCR domain of CD163, more preferably for any one of SRCR domains 1-9 of CD163 (e.g. for human CD163: amino acids 51-152, 159-259, 266-366, 373-473, 478-578, 583-683, 719-819, 824-926 and 929-1029, respectively, of UniProt Q86VB7, sequence version 2), even more preferably for any one of SRCR domains 1-3 of CD163 (e.g. for human CD163: amino acids 51-152, 159-259, 266-366, and 373-473, respectively, of UniProt Q86VB7, sequence version 2), especially for SRCR domain 1 of CD163 (in particular with the amino acid sequence of any one of SEQ ID NOs: 1-8 of WO 2011/039510 A2, especially SEQ ID NO: 1 of WO 2011/039510 A2).

In a particular preference, the anti-CD163 antibody or CD163-binding fragment thereof is capable of competing for binding to (preferably human) CD163 with a (preferably human) hemoglobin-haptoglobin complex (e.g. in an ELISA).

In another particular preference, the anti-CD163 antibody or CD163-binding fragment thereof is capable of competing for binding to human CD163 with any of the anti-human CD163 mAbs disclosed herein, in particular Mac2-48 or Mac2-158 as disclosed in WO 2011/039510 A2.

In yet another particular preference, the anti-CD163 antibody or CD163-binding fragment thereof is capable of competing for binding to human CD163 with an antibody having a heavy chain variable (V_{H}) region consisting of the amino acid sequence

(SEQ ID NO: 137)
DVQLQESGPGLVKPSQSLSLTCTVTGYSITSDYAWNWIRQFPGNKLEWM
GYITYSGITNYNPSLKSQISITRDTSKNQFFLQLNSVTTEDTATYYCVS
GTYYFDYWGQGTTLTVSS,

and having a light-chain variable (VL) region consisting of the amino acid sequence

(SEQ ID NO: 138)
SVVMTQTPKSLLISIGDRVTITCKASQSVSSDVAWFQQKPGQSPKPLIY
YASNRYTGVPDRFTGSGYGTDFTFTISSVQAEDLAVYFCGQDYTSPRTF
GGGTKLEIKRA (e.g. in an ELISA).

Details on competitive binding experiments are known to the person of skilled in the art (e.g. based on ELISA) and are for instance disclosed in WO 2011/039510 A2 (which is included herein by reference).

In the course of the present invention, the epitopes of antibodies E10B10 and Mac2-158 as disclosed in WO 2011/039510 were mapped by fine mapping using circular peptide arrays, whereby the peptides were derived from CD163. These epitopes are particularly suitable for binding of the anti-CD163 antibody (or CD163-binding fragment thereof) of the inventive compound.

Accordingly, in particularly preferred embodiment, the anti-CD163 antibody or CD163-binding fragment thereof is specific for peptide consisting of 7-25, preferably 8-20, even more preferably 9-15, especially 10-13 amino acids, wherein the peptide comprises the amino acid sequence CSGRVEVKVQEEWGTVCNNGWSMEA (SEQ ID NO: 139) or a 7-24 amino--acid fragment thereof. Preferably, this peptide comprises the amino acid sequence GRVEVKVQEEW (SEQ ID NO: 140), WGTVCNNGWS (SEQ ID NO: 141) or WGTVCNNGW (SEQ ID NO: 142). More preferably, the peptide comprises an amino acid sequence selected from EWGTVCNNGWSME (SEQ ID NO: 143), QEEWGTVCNNGWS (SEQ ID NO: 144), WGTVCNNGWSMEA (SEQ It) NO : 145), EEWGTVCNNGWSM (SEQ ID NO: 146), VQEEWGTVCNNGW (SEQ ID NO: 147), EWGTVCNNGW (SEQ ID NO: 148) and WGTVCNNGWS (SEQ ID NO: 141). Even more preferably, the peptide consists of an amino acid sequence selected from EWGTVCNNGWSME (SEQ ID NO: 143), QEEWGTVCNNGWS (SEQ It) NO: 144.), WGTVCNNGWSMEA. (SEQ ID NO: 145) , EEWGTVCNNGWSM (SEQ ID NO: 146) , VQEEWGTVCNNGW (SEQ ID NO: 147), EWGTVCNNCW (SEQ ID NO: 148) and WGTVCNNGWS (SEQ ID NO: 141), optionally with an N-terminal and/or C-terminal cysteine residue.

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence DHVSCRGNESALWDCKHDGWG (SEQ ID NO: 149) or a 7-20 amino-acid fragment thereof. Preferably, this peptide comprises the amino acid sequence ESALW (SEQ ID NO: 150) or. ALW. More preferably, the peptide comprises an amino acid sequence selected from ESALWDC (SEQ ID NO: 151), RGNESALWDC (SEQ ID NO: 152), SCRGNESALW (SEQ ID NO: 153), VSCRGNESALWDC (SEQ ID NO: 154), ALWDCKHDGW (SEQ II) NO: 155), DHVSCRGNESALW (SEQ ID NO: 156), CRGNESALWD (SEQ ID NO: 157), NESALWDCKHDGW (SEQ ID NO: 158) and ESALWDCKHDGWG (SEQ ID NO: 159). Even more preferably, the peptide consists of an amino acid sequence selected from. ESALWDC (SEQ ID NO: 151), RGNESALWDC (SEQ ID NO: 152), SCRGNESALW (SEQ ID NO: 153), VSCRGNESALWDC (SEQ. ID NO: 154), ALWDCKHDGW (SEQ ID NO: 155), DHVSCRGNESALW (SEQ. ID NO: 156), CRGNESALWD (SEQ ID NO: 157), NESALWDCKHDGW (SEQ ID NO: 158) and ESALWDCKEDGWG (SFQ ID O: 159) , optionally with an N-terminal and/or C-terminal cysteine residue.

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence SSLGGTDKELRLVDGENKCS (SEQ ID NO: 160) or a 7-19 amino-acid fragment thereof. Preferably, this peptide comprises the amino acid sequence SSLGGTDKELR (SEQ ID NO: 161) or SSLGG (SEQ ID NO: 162). More preferably, the peptide comprises an amino acid sequence selected from SSLGGTDKELR (SEQ ID NO: 161), SSLGGTDKEL (SEQ. ID NO: 163), SSLGGTDKE (SEQ ID NO: 164), SSLGGTDK (SEQ ID NO: 165), SSLGGTD (SEQ ID NO: 166), SSLGGT (SEQ ID NO: 167) and SSLGG (SEQ ID NO: 162). Even more preferably, the peptide consists of an amino acid sequence selected from SSLGGTDKELR (SEQ ID NO: 161), SSLGGTDKEL (SEQ ID NO: 163), SSLGGTDKE (SEQ ID NO: 164), SSLGGTDK (SEQ ID NO: 165) , SSLGGTD (SEQ ID NO: 166), SSLGGT (SEQ ID NO: 167) and SSLGG (SEQ ID NO: 162), optionally with an N-terminal and/or C-terminal cysteine residue.

The peptides (or peptide n-mers) are preferably covalently conjugated (or covalently bound) to the biopolymer scaffold via a (non-immunogenic) linker known in the art such as for example amine-to-sulfhydryl linkers and bifunctional NHS-PEG-maleimide linkers or other linkers known in the art. Alternatively, the peptides (or peptide n-mers) can be bound to the epitope carrier scaffold e.g. by formation of a disulfide bond between the protein and the peptide (which is also referred to as “linker” herein), or using non-covalent assembly techniques, spontaneous isopeptide bond formation or unnatural amino acids for bio-orthogonal chemistry via genetic code expansion techniques (reviewed by Howarth et al 2018 and Lim et al 2016).

The compound of the present invention may comprise e.g. at least two, preferably between 3 and 40 copies of one or several different peptides (which may be present in different forms of peptide n-mers as disclosed herein). The compound may comprise one type of epitopic peptide (in other words: antibody-binding peptide or paratope-binding peptide), however the diversity of epitopic peptides bound to one biopolymer scaffold molecule can be a mixture of e.g. up to 8 different epitopic peptides.

Typically, since the peptides present in the inventive compound specifically bind to selected undesired antibodies, their sequence is usually selected and optimized such that they provide specific binding in order to guarantee selectivity of undesired antibody depletion from the blood. For this purpose, the peptide sequence of the peptides typically corresponds to the entire epitope sequence or portions of the undesired antibody epitope. The peptides used in the present invention can be further optimized by exchanging one, two or up to three amino-acid positions, allowing e.g. for modulating the binding affinity to the undesired antibody that needs to be depleted. Such single or multiple amino-acid substitution strategies that can provide “mimotopes” with increased binding affinity and are known in the field and were previously developed using phage display strategies or peptide microarrays. In other words, the peptides used in the present invention do not have to be completely identical to the native epitope sequences of the undesired antibodies.

Typically, the peptides used in the compound of the present invention (e.g. peptide P or Pₐ or P_{b} or P₁ or P2) are composed of one or more of the 20 amino acids commonly present in mammalian proteins. In addition, the amino acid repertoire used in the peptides may be expanded to post-translationally modified amino acids e.g. affecting antigenicity of proteins such as post translational modifications, in particular oxidative post translational modifications (see e.g. Ryan 2014) or modifications to the peptide backbone (see e.g. Muller 2018), or to non-natural amino acids (see e.g. Meister et al 2018). These modifications may also be used in the peptides e.g. to adapt the binding interaction and specificity between the peptide and the variable region of an undesired antibody. In particular, epitopes (and therefore the peptides used in the compound of the present invention) can also contain citrulline as for example in autoimmune diseases. Furthermore, by introducing modifications into the peptide sequence the propensity of binding to an HLA molecule may be reduced, the stability and the physicochemical characteristics may be improved or the affinity to the undesired antibody may be increased.

In many cases, the undesired antibody that is to be depleted is oligo- or polyclonal (e.g. autoantibodies, ADAs or alloantibodies are typically poly- or oligoclonal), implying that undesired (polyclonal) antibody epitope covers a larger epitopic region of a target molecule. To adapt to this situation, the compound of the present invention may comprise a mixture of two or several epitopic peptides (in other words: antibody-binding peptides or paratope-binding peptides), thereby allowing to adapt to the polyclonality or oligoclonality of an undesired antibody.

Such poly-epitopic compounds of the present invention can effectively deplete undesired antibodies and are more often effective than mono-epitopic compounds in case the epitope of the undesired antibody extends to larger amino acid sequence stretches.

It is advantageous if the peptides used for the inventive compound are designed such that they will be specifically recognized by the variable region of the undesired antibodies to be depleted. The sequences of peptides used in the present invention may e.g. be selected by applying fine epitope mapping techniques (i.e. epitope walks, peptide deletion mapping, amino acid substitution scanning using peptide arrays such as described in Carter et al 2004, and Hansen et al 2013) on the undesired antibodies.

It is highly preferred that the peptides used for the inventive compound do not bind to any HLA Class I or HLA Class II molecule (i.e. of the individual to be treated, e.g. human), in order to prevent presentation and stimulation via a T-cell receptor in vivo and thereby induce an immune reaction. It is generally not desired to involve any suppressive (or stimulatory) T-cell reaction in contrast to antigen-specific immunologic tolerization approaches. Therefore, to avoid T-cell epitope activity as much as possible, the peptides of the compound of the present invention (e.g. peptide P or Pₐ or P_{b} or P₁ or P₂) preferably fulfil one or more of the following characteristics:
To reduce the probability for a peptide used in the compound of the present invention to bind to an HLA Class II or Class I molecule, the peptide (e.g. peptide P or Pₐ or P_{b} or P₁ or P2) has a preferred length of 6-13 amino acids.
To further reduce the probability that such a peptide binds to an HLA Class II or Class I molecule, it is preferred to test the candidate peptide sequence by HLA binding prediction algorithms such as NetMHCII-2.3 (reviewed by Jensen et al 2018). Preferably, a peptide (e.g. peptide P or Pₐ or P_{b} or P₁ or P₂) used in the compound of the present invention has (predicted) HLA binding (IC50) of at least 500 nM. More preferably, HLA binding (IC50) is more than 1000 nM, especially more than 2000 nM (cf. e.g. Peters et al 2006). In order to decrease the likelihood of HLA Class I binding, NetMHCpan 4.0 may also be applied for prediction (Jurtz et al 2017).
To further reduce the probability that such a peptide binds to an HLA Class I molecule, the NetMHCpan Rank percentile threshhold can be set to a background level of 10% according to Koşaloğlu-Yalçin et al 2018. Preferably, a peptide (e.g. peptide P or Pₐ or P_{b} or P₁ or P₂) used in the compound of the present invention therefore has a % Rank value of more than 3, preferably more than 5, more preferably more than 10 according to the NetMHCpan algorithm.
To further reduce the probability that such a peptide binds to an HLA Class II molecule, it is beneficial to perform in vitro HLA-binding assays commonly used in the art such as for example refolding assays, iTopia, peptide rescuing assays or array-based peptide binding assays. Alternatively, or in addition thereto, LC-MS based analytics can be used, as e.g. reviewed by Gfeller et al 2016.

For stronger reduction of the titre of the undesired antibodies, it is preferred that the peptides used in the present invention are circularized (see also Example 4). Accordingly, in a preferred embodiment, at least one occurrence of P is a circularized peptide. Preferably at least 10% of all occurrences of P are circularized peptides, more preferably at least 25% of all occurrences of P are circularized peptides, yet more preferably at least 50% of all occurrences of P are circularized peptides, even more preferably at least 75% of all occurrences of P are circularized peptides, yet even more preferably at least 90% of all occurrences of P are circularized peptides or even at least 95% of all occurrences of P are circularized peptides, especially all of the occurrences of P are circularized peptides. Several common techniques are available for circularization of peptides, see e.g. Ong et al 2017. It goes without saying that “circularized peptide” as used herein shall be understood as the peptide itself being circularized, as e.g. disclosed in Ong et al. (and not e.g. grafted on a circular scaffold with a sequence length that is longer than 13 amino acids). Such peptides may also be referred to as cyclopeptides herein.

Further, for stronger reduction of the titre of the undesired antibodies relative to the amount of scaffold used, in a preferred embodiment of the compound of the present invention, independently for each of the peptide n-mers, n is at least 2, more preferably at least 3, especially at least 4. Usually, in order to avoid complexities in the manufacturing process, independently for each of the peptide n-mers, n is less than 10, preferably less than 9, more preferably less than 8, even more preferably less than 7, yet even more preferably less than 6, especially less than 5. To benefit from higher avidity through divalent binding of the undesired antibody, it is highly preferred that, for each of the peptide n-mers, n is 2.

For multivalent binding of the undesired antibodies, it is advantageous that the peptide dimers or n-mers are spaced by a hydrophilic, structurally flexible, immunologically inert, non-toxic and clinically approved spacer such as (hetero-) bifunctional and -trifunctional polyethylene glycol (PEG) spacers (e.g. NHS-PEG-Maleimide) - a wide range of PEG chains is available and PEG is approved by the FDA. Alternatives to PEG linkers such as immunologically inert and non-toxic synthetic polymers or glycans are also suitable. Accordingly, in the context of the present invention, the spacer (e.g. spacer S) is preferably selected from PEG molecules or glycans. For instance, the spacer such as PEG can be introduced during peptide synthesis. Such spacers (e.g. PEG spacers) may have a molecular weight of e.g. 10000 Dalton. Evidently, within the context of the present invention, the covalent binding of the peptide n-mers to the biopolymer scaffold via a linker each may for example also be achieved by binding of the linker directly to a spacer of the peptide n-mer (instead of, e.g., to a peptide of the peptide n-mer).

Preferably, each of the peptide n-mers is covalently bound to the biopolymer scaffold, preferably via a linker each.

As used herein, the linker may e.g. be selected from disulphide bridges and PEG molecules.

According to a further preferred embodiment of the inventive compound, at least one occurrence of P is Pₐ and/or at least one occurrence of P is P_{b} (wherein Pₐ and P_{b} each independently is a peptide as defined above for P and/or P₁ and P₂). Preferably, independently for each occurrence, P is Pₐ or P_{b}.

Furthermore, it is preferred when in the first peptide n-mer, each occurrence of P is Pₐ and, in the second peptide n-mer, each occurrence of P is P_{b}. Alternatively, or in addition thereto, Pₐ and/or P_{b} is circularized.

Divalent binding is particularly suitable to reduce antibody titres. According, in a preferred embodiment,
the first peptide n-mer is Pa- S - Pₐ and the second peptide n-mer is Pₐ-S-Pₐ;
the first peptide n-mer is Pa-S-Pₐ and the second peptide n-mer is P_{b}-S-P_{b};
the first peptide n-mer is P_{b} -S-P_{b} and the second peptide n-mer is P_{b}-S-P_{b};
the first peptide n-mer is Pa-S-P_{b} and the second peptide n-mer is Pₐ-S-P_{b};
the first peptide n-mer is Pa-S-P_{b} and the second peptide n-mer is Pₐ-S-Pₐ;or
the first peptide n-mer is Pa-S-P_{b} and the second peptide n-mer is P_{b}-S-P_{b}.

For increasing effectivity, in particular in autoimmune disease (which is usually based on polyclonal antibodies, see above), in a preferred embodiment the first peptide n-mer is different from the second peptide n-mer. For similar reasons, preferably, the peptide Pₐ is different from the peptide P_{b}, preferably wherein the peptide Pₐ and the peptide P_{b} are two different epitopes of the same antigen or two different epitope parts of the same epitope.

Especially for better targeting of polyclonal antibodies, it is advantageous when the peptide Pₐ and the peptide P_{b} comprise the same amino-acid sequence fragment, wherein the amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.

Further, for stronger reduction of the titre of the undesired antibodies relative to the amount of scaffold used, the compound comprises a plurality of said first peptide n-mer (e.g. up to 10 or 20 or 30) and/or a plurality of said second peptide n-mer (e.g. up to 10 or 20 or 30).

For stronger reduction of the titre of the undesired antibodies relative to the amount of scaffold used, the compound may also comprise at least
a third peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is P_{c}, wherein P_{c} is a peptide defined as disclosed herein above (e.g. for P, P₁, P₂, and/or Pₐ),
more preferably wherein P_{c} is circularized; preferably a fourth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is P_{d}, wherein Pd is a peptide defined as disclosed herein above (e.g. for P, P₁, P₂, and/or Pₐ),
more preferably wherein P_{d} is circularized; preferably a fifth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pₑ, wherein Pe is a peptide defined as disclosed herein above (e.g. for P, P₁, P₂, and/or Pₐ), more preferably wherein Pₑ is circularized; preferably a sixth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pf, wherein Pf is a peptide defined as disclosed herein above (e.g. for P, P₁, P₂, and/or Pₐ), more preferably wherein P_{f} is circularized; preferably a seventh peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pg, wherein Pg is a peptide defined as disclosed herein above (e.g. for P, P₁, P₂, and/or Pₐ),
more preferably wherein P_{g} is circularized; preferably an eigth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pₕ, wherein Pₕ is a peptide defined as disclosed herein above (e.g. for P, P₁, P₂, and/or Pₐ),
more preferably wherein Ph is circularized; preferably a ninth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pᵢ, wherein Pᵢ is a peptide defined as disclosed herein above (e.g. for P, P₁, P₂, and/or Pₐ),
more preferably wherein Pi is circularized; preferably a tenth peptide n-mer of the general formula:

P (-S-P)₍ₙ₋₁₎,
wherein, independently for each occurrence, P is a peptide defined as disclosed herein above (e.g. for P, P₁, P₂, and/or Pₐ), and S is a non-peptide spacer,
preferably wherein each occurrence of P is Pⱼ, wherein is a peptide defined as disclosed herein above (e.g. for P, P₁, P₂, and/or Pₐ),
more preferably wherein Pⱼ is circularized.

Peptides P_{c}-Pⱼ may have one or more of same features (e.g. sequence) as disclosed herein for peptides Pₐ and P_{b} (and/or for peptides P, P₁, P₂). All preferred features disclosed herein for P, P₁, and P₂, are also preferred features of the peptides Pₐ-Pⱼ. As also illustrated above, it is highly preferred when the compound of the present invention is non-immunogenic in a mammal, preferably in a human, in a non-human primate, in a sheep, in a pig, in a dog or in a rodent.

In the context of the present invention, a non-immunogenic compound preferably is a compound wherein the biopolymer scaffold (if it is a protein) and/or the peptides (of the peptide n-mers) have an IC50 higher than 100 nM, preferably higher than 500 nM, even more preferably higher than 1000 nM, especially higher than 2000 nM, against HLA-DRB1 0101 as predicted by the NetMHCII-2.3 algorithm. The NetMHCII-2.3 algorithm is described in detail in Jensen et al, which is incorporated herein by reference. The algorithm is publicly available under http://www.cbs.dtu.dk/services/NetMHCII-2.3/. Even more preferably, a non-immunogenic compound (or pharmaceutical composition) does not bind to any HLA and/or MHC molecule (e.g. in a mammal, preferably in a human, in a non-human primate, in a sheep, in a pig, in a dog or in a rodent; or of the individual to be treated) in vivo.

According to a further preference, the compound is for intracorporeal sequestration (or intracorporeal depletion) of at least one antibody in an individual, preferably in the bloodstream of the individual and/or for reduction of the titre of at least one antibody in the individual, preferably in the bloodstream of the individual. Preferably the antibody is an anti-factor VIII antibody, preferably an anti-human factor VIII antibody.

In an aspect, the present invention relates to a pharmaceutical composition comprising the inventive compound and at least one pharmaceutically acceptable excipient.

In embodiments, the composition is prepared for intraperitoneal, subcutaneous, intramuscular and/or intravenous administration. In particular, the composition is for repeated administration (since it is typically non-immunogenic).

In a preference, the molar ratio of peptides (e.g. P or Pₐ or P_{b}) to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

In a further preferred embodiment, the pharmaceutical composition further comprises a factor VIII replacement product. Preferably said factor VIII replacement product is factor VIII (or antihemophilic factor), most preferably human factor VIII. Such factor VIII replacement products are known e.g. under the brand names Hemofil-M, Koate-DVI, and Monoclate-P.

In another aspect, the compound and/or the pharmaceutical composition of the present invention is for use in therapy.

Preferably, the compound and/or the pharmaceutical composition is for use in prevention or treatment of of hemophilia A in an individual. Preferably the hemophilia A is congenital hemophilia A and/or acquired hemophilia A.

In a preferred embodiment, the individual further receives factor VIII replacement therapy.

In the course of the present invention, it turned out that the in vivo kinetics of undesirable-antibody lowering by the inventive compound is typically very fast, sometimes followed by a mild rebound of the undesirable antibody. It is thus particularly preferred when the compound (or the pharmaceutical composition comprising the compound) is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window; in particular wherein this window is followed by administration of the factor VIII replacement product as described herein within 24 hours, preferably within 12 hours (but typically after at least 6 hours). For instance, the pharmaceutical composition may be administered at −24 hrs and −12 hrs before administration of the factor VIII replacement product at 0 hrs.

In a preferred embodiment, the inventive compound is administered to the individual, and a factor VIII replacement product (preferably factor VIII, most preferably human factor VIII) is administered to the individual in combination with said compound, before said compound is administered, or after said compound has been administered, preferably wherein said composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window; in particular wherein this window is followed by administration of the factor VIII replacement product within 24 hours, preferably within 12 hours. The factor VIII replacement product may also be present in the same composition as the inventive compound (i.e. in the inventive pharmaceutical composition). It is preferred that the inventive pharmaceutical composition (comprising the inventive compound and optionally also comprising a factor VIII replacement product) is administered to the individual, and a factor VIII replacement product (preferably factor VIII, most preferably human factor VIII) is administered to the individual in combination with said composition, before said composition is administered, or after said composition has been administered.

In particular, the inventive compound (or the pharmaceutical composition comprising the compound) is for use in inhibiting neutralization and/or inhibition of a factor VIII replacement product, preferably factor VIII, more preferably human factor VIII, in an individual, preferably wherein the compound (or the pharmaceutical composition) is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window; in particular wherein this window is followed by administration of the factor VIII replacement product within 24 hours, preferably within 12 hours.

In embodiments, one or more antibodies are present in the individual which are specific for at least one occurrence of the peptide of the inventive compound (e.g. the peptide P, P₁, P₂, or for peptide Pₐ and/or peptide P_{b}), preferably wherein said antibodies are specific for factor VIII.

It is highly preferred that the composition is non-immunogenic in the individual (e.g. it does not comprise an adjuvant or an immunostimulatory substance that stimulates the innate or the adaptive immune system, e.g. such as an adjuvant or a T-cell epitope).

The composition of the present invention may be administered at a dose of 1-1000 mg, preferably 2-500 mg, more preferably 3-250 mg, even more preferably 4-100 mg, especially 5-50 mg, compound per kg body weight of the individual, preferably wherein the composition is administered repeatedly. Such administration may be intraperitoneally, subcutaneously, intramuscularly or intravenously.

In an aspect, the present invention relates to a method of ameliorating or treating hemophilia A in an individual in need thereof, comprising
obtaining the inventive pharmaceutical composition; and
administering an effective amount of the pharmaceutical composition to the individual. All preferred features disclosed for the compound and/or the pharmaceutical composition for use in prevention or treatment of of hemophilia A in an individual also apply to this method.

In a further aspect, the present invention relates to a method of sequestering (or depleting) one or more antibodies present in an individual, comprising
obtaining a pharmaceutical composition as defined herein, wherein the composition is non-immunogenic in the individual and wherein the one or more antibodies present in the individual are specific for at least one occurrence of P, or for peptide Pₐ and/or peptide P_{b}; and
administering (in particular repeatedly administering, e.g. at least two times, preferably at least three times, more preferably at least five times) the pharmaceutical composition to the individual.

In a preference, the one or more antibodies are specific for factor VIII, preferably human factor VIII.

Preferably, the biopolymer scaffold is autologous with respect to the individual, preferably wherein the biopolymer scaffold is an autologous protein (i.e. murine albumin is used when the individual is a mouse).

In an embodiment, the individual is administered a factor VIII replacement product, and the one or more antibodies present in the individual are specific for said factor VIII replacement product, preferably wherein said administering of the factor VIII replacement product is prior to, concurrent with and/or subsequent to said administering of the pharmaceutical composition. Preferably the factor VIII replacement product is factor VIII, preferably human factor VIII.

In a further aspect, the present invention relates to a peptide, wherein the peptide is defined as disclosed herein for any one of the at least two peptides of the inventive compound, P, P₁, P₂, Pₐ, or P_{b}.

In certain embodiments, such peptides may be used as probes for the diagnostic typing and analysis of neutralizing antibodies against factor VIII, such as anti-drug antibodies induced by substitution therapies, or gene therapies, or by any other circulating (auto-)antibodies against factor VIII such as in acquired hemophilia A. The peptides can e.g. be used as part of a diagnostic anti-factor VIII typing or screening device or kit or procedure, as a companion diagnostic, for patient stratification or for monitoring neutralizing antibody levels in the course of therapeutic treatments.

In a further aspect, the invention relates to a method for detecting and/or quantifying anti-factor VIII antibodies in a biological sample comprising the steps of
bringing the sample into contact with the peptide defined as disclosed herein (e.g. for P, P1, P₂, Pₐ, or P_{b}) , and
detecting the presence and/or concentration of anti-factor VIII antibodies in the sample.

The skilled person is familiar with methods for detecting and/or quantifying antibodies in biological samples. The method can e.g. be a sandwich assay, preferably an enzyme-linked immunosorbent assay (ELISA), or a surface plasmon resonance (SPR) assay.

In a preference, the peptide is immobilized on a solid support, preferably an ELISA plate or an SPR chip or a biosensor-based diagnostic device with an electrochemical, fluorescent, magnetic, electronic, gravimetric or optical biotransducer. Alternatively, or in addition thereto, the peptide may be coupled to a reporter or reporter fragment, such as a reporter fragment suitable for a protein-fragment complementation assay (PCA); see e.g. Li et al, 2019, or Kanulainen et al, 2021.

Preferably, the sample is obtained from a mammal, preferably a human. Preferably the sample is a blood sample, preferably a whole blood, serum, or plasma sample.

The invention further relates to the use of a peptide defined as disclosed herein (e.g. for P, P₁, P₂, Pₐ, or P_{b}) in a diagnostic assay, preferably ELISA, preferably as disclosed herein above.

A further aspect of the invention relates to a diagnostic device comprising the peptide defined as disclosed herein (e.g. for P, P₁, P₂, Pₐ, or P_{b}) , preferably immobilized on a solid support. In a preference, the solid support is an ELISA plate or a surface plasmon resonance chip. In another preference, the diagnostic device is a biosensor-based diagnostic device with an electrochemical, fluorescent, magnetic, electronic, gravimetric or optical biotransducer.

In another preferred embodiment, the diagnostic device is a lateral flow assay.

The invention further relates to a diagnostic kit comprising a peptide defined as disclosed herein (e.g. for P, P₁, P₂, Pₐ, or P_{b}), preferably a diagnostic device as defined herein. Preferably the diagnostic kit further comprises one or more selected from the group of a buffer, a reagent, instructions. Preferably the diagnostic kit is an ELISA kit.

A further aspect relates to an apheresis device comprising the peptide defined as disclosed herein (e.g. for P, P₁, P₂, Pₐ, or P_{b}). Preferably the peptide is immobilized on a solid carrier. It is especially preferred if the apheresis device comprises at least two, preferably at least three, more preferably at least four different peptides defined as disclosed herein (e.g. for P, P₁, P₂, Pₐ, or P_{b}) . In a preferred embodiment the solid carrier comprises the inventive compound.

Preferably, the solid carrier is capable of being contacted with blood or plasma flow. Preferably, the solid carrier is a sterile and pyrogen-free column.

In the context of the present invention, for improved bioavailability, it is preferred that the inventive compound has a solubility in water at 25° C. of at least 0.1 μg/ml, preferably at least 1 μg/ml, more preferably at least 10 μg/ml, even more preferably at least 100 μg/ml, especially at least 1000 μg/ml.

The term “preventing” or “prevention” as used herein means to stop a disease state or condition from occurring in a patient or subject completely or almost completely or at least to a (preferably significant) extent, especially when the patient or subject or individual is predisposed to such a risk of contracting a disease state or condition.

The pharmaceutical composition of the present invention is preferably provided as a (typically aqueous) solution, (typically aqueous) suspension or (typically aqueous) emulsion. Excipients suitable for the pharmaceutical composition of the present invention are known to the person skilled in the art, upon having read the present specification, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. Other suitable excipients include any compound that does not itself induce the production of antibodies in the patient (or individual) that are harmful for the patient (or individual). Examples are well tolerable proteins, polysaccharides, polylactic acids, polyglycolic acid, polymeric amino acids and amino acid copolymers. This pharmaceutical composition can (as a drug) be administered via appropriate procedures known to the skilled person (upon having read the present specification) to a patient or individual in need thereof (i.e. a patient or individual having or having the risk of developing the diseases or conditions mentioned herein). The preferred route of administration of said pharmaceutical composition is parenteral administration, in particular through intraperitoneal, subcutaneous, intramuscular and/or intravenous administration. For parenteral administration, the pharmaceutical composition of the present invention is preferably provided in injectable dosage unit form, e.g. as a solution (typically as an aqueous solution), suspension or emulsion, formulated in conjunction with the above-defined pharmaceutically acceptable excipients. The dosage and method of administration, however, depends on the individual patient or individual to be treated. Said pharmaceutical composition can be administered in any suitable dosage known from other biological dosage regimens or specifically evaluated and optimised for a given individual. For example, the active agent may be present in the pharmaceutical composition in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/kg body weight (per administration session). The administration may occur e.g. once daily, once every other day, once per week or once every two weeks. As the preferred mode of administration of the inventive pharmaceutical composition is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises or consists of 0.1-990 μg, preferably 1-900 μg, more preferably 10-200 μg compound, and option-ally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

It is evident to the skilled person that active agents and drugs described herein can also be administered in salt-form (i.e. as a pharmaceutically acceptable salt of the active agent). Accordingly, any mention of an active agent herein shall also include any pharmaceutically acceptable salt forms thereof.

Methods for chemical synthesis of peptides used for the compound of the present invention are well-known in the art. Of course, it is also possible to produce the peptides using recombinant methods. The peptides can be produced in microorganisms such as bacteria, yeast or fungi, in eukaryotic cells such as mammalian or insect cells, or in a recombinant virus vector such as adenovirus, poxvirus, herpesvirus, Simliki forest virus, baculovirus, bacteriophage, sindbis virus or sendai virus. Suitable bacteria for producing the peptides include E. coli, B. subtilis or any other bacterium that is capable of expressing such peptides. Suitable yeast cells for expressing the peptides of the present invention include Saccharomyces cerevisiae, Schizosaccharomyces pombe, Candida, Pichiapastoris or any other yeast capable of expressing peptides. Corresponding means and methods are well known in the art. Also, methods for isolating and purifying recombinantly produced peptides are well known in the art and include e.g. gel filtration, affinity chromatography, ion exchange chromatography etc.

Beneficially, cysteine residues are added to the peptides at the N- and/or C-terminus to facilitate coupling to the biopolymer scaffold, especially.

To facilitate isolation of said peptides, fusion polypeptides may be made wherein the peptides are translationally fused (covalently linked) to a heterologous polypeptide which enables isolation by affinity chromatography. Typical heterologous polypeptides are His-Tag (e.g. His6; 6 histidine residues), GST-Tag (Glutathione-S-transferase) etc. The fusion polypeptide facilitates not only the purification of the peptides but can also prevent the degradation of the peptides during the purification steps. If it is desired to remove the heterologous polypeptide after purification, the fusion polypeptide may comprise a cleavage site at the junction between the peptide and the heterologous polypeptide. The cleavage site may consist of an amino acid sequence that is cleaved with an enzyme specific for the amino acid sequence at the site (e.g. proteases).

The coupling/conjugation chemistry used to link the peptides/peptide n-mers to the biopolymer scaffold (e.g. via heterobifunctional compounds such as GMBS and of course also others as described in “Bioconjugate Techniques”, Greg T. Hermanson) or used to conjugate the spacer to the peptides in the context of the present invention can also be selected from reactions known to the skilled in the art. The biopolymer scaffold itself may be recombinantly produced or obtained from natural sources.

In the context of all aspects of the present invention, factor VIII preferably is human factor VIII, most preferably factor VIII identified by UniProt accession code P00451.

In the context of all aspects of the present invention, hemophilia A preferably refers to a factor VIII deficiency. Preferably hemophilia A is congenital hemophilia A and/or acquired hemophilia A.

A “factor VIII replacement product” as referred to in the context of the present application preferably is factor VIII, especially human factor VIII. Preferably, the factor VIII replacement product is recombinant (human) factor VIII. Factor VIII may also be referred to as antihemophilic factor. Factor VIII replacement products are known e.g. under the brand names Hemofil-M, Koate-DVI, and Monoclate-P.

Herein, the term “specific for” —as in “molecule A specific for molecule B”—means that molecule A has a binding preference for molecule B compared to other molecules in an individual's body. Typically, this entails that molecule A (such as an antibody) has a dissociation constant (also called “affinity”) in regard to molecule B (such as the antigen, specifically the binding epitope thereof) that is lower than (i.e. “stronger than”) 1000 nM, preferably lower than 100 nM, more preferably lower than 50 nM, even more preferably lower than 10 nM, especially lower than 5 nM.

Herein, “UniProt” refers to the Universal Protein Resource. UniProt is a comprehensive resource for protein sequence and annotation data. UniProt is a collaboration between the European Bioinformatics Institute (EMBL-EBI), the SIB Swiss Institute of Bioinformatics and the Protein Information Resource (PIR). Across the three institutes more than 100 people are involved through different tasks such as database curation, software development and support. Website: https://www.uniprot.org/Entries in the UniProt databases are identified by their accession codes (referred to herein e.g. as “UniProt accession code” or briefly as “UniProt” followed by the accession code), usually a code of six alphanumeric letters (e.g. “Q1HVF7”). If not specified otherwise, the accession codes used herein refer to entries in the Protein Knowledgebase (UniProtKB) of UniProt. If not stated otherwise, the UniProt database state for all entries referenced herein is of 22 Sep. 2020 (UniProt/UniProtKB Release 2020_04).

In the context of the present application, sequence variants (designated as “natural variant” in UniProt) are expressly included when referring to a UniProt database entry.

“Percent (%) amino acid sequence identity” or “X% identical” (such as “70% identical”) with respect to a reference polypeptide or protein sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, Megalign (DNASTAR) or the “needle” pairwise sequence alignment application of the EMBOSS software package. Those skilled in the art can determine appropriate parameters for aligning sequences, including any algorithms needed to achieve maximal alignment over the full length of the sequences being compared. For purposes herein, however, % amino acid sequence identity values are calculated using the sequence alignment of the computer programme “needle” of the EMBOSS software package (publicly available from European Molecular Biology Laboratory; Rice et al., EMBOSS: the European Molecular Biology Open Software Suite, Trends Genet. 2000 June;16(6):276-7) .

The needle programme can be accessed under the web site http://www.ebi.ac.uk/Tools/psa/embossneedle/ or downloaded for local installation as part of the EMBOSS package from http://emboss.sourceforge.net/. It runs on many widely-used UNIX operating systems, such as Linux.

To align two protein sequences, the needle programme is preferably run with the following parameters:

Commandline: needle-auto-stdout-asequence SEQUENCE_FILE_A -bsequence SEQUENCE_FILE_B -datafile EBLOSUM62-gapopen 10.0 -gapextend 0.5-endopen 10.0-endextend 0.5-aformat3 pair-sprotein1-sprotein2 (Align_format: pair Report_file: stdout)

The % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program needle in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. In cases where “the sequence of A is more than N% identical to the entire sequence of B”, Y is the entire sequence length of B (i.e. the entire number of amino acid residues in B). Unless specifically stated otherwise, all % amino acid sequence identity values used herein are obtained as described in the immediately preceding paragraph using the needle computer program.

The present invention further relates to the following embodiments:

Embodiment 1. A compound comprising a biopolymer scaffold and at least two peptides, preferably derived from (human) factor VIII, with a sequence length of 6-13 amino acids,
wherein each of the peptides independently comprises a 6-amino-acid fragment, preferably a 7-, more preferably an 8-, even more preferably a 9-, even more preferably a 10-, even more preferably an 11-, yet even more preferably a 12-, most preferably a 13-amino-acid fragment, of the amino acid sequence of (preferably human) factor VIII, preferably as identified by UniProt accession code P00451,
optionally wherein at most three, preferably at most two, more preferably at most one amino acid is independently substituted by any other amino acid.

Embodiment 2. A compound, preferably the compound of embodiment 1, comprising a biopolymer scaffold and at least two peptides, preferably derived from (human) factor VIII, with a sequence length of 6-13 amino acids,
wherein each of the peptides independently comprises a 6-amino-acid fragment, preferably a 7-, more preferably an 8-, even more preferably a 9-, even more preferably a 10-, even more preferably an 11-, yet even more preferably a 12-, most preferably a 13-amino-acid fragment, of an amino-acid sequence selected from the group consisting of:
STLRMELMGCDLNSCSMP (SEQ ID NO: 1), IALRMEVLGCEAQDLY (SEQ ID NO: 2), QYLNNGPQRIGRKYKKVRFM (SEQ ID NO: 3), LYGEVGDTLLIIFK (SEQ ID NO: 4), NGPQRIGRKYKKVRFM (SEQ ID NO: 5), KSQYLNNGPQRIGRK (SEQ ID NO: 6), PHGITDVRPLYSRRLP (SEQ ID NO: 7), THYSIRSTLR (SEQ ID NO: 8), KARLHLQGRSNAWRP (SEQ ID NO: 9), QDGHQWTLFF (SEQ ID NO: 10), NSLDPPLLTRYLRIH (SEQ ID NO: 11), IHPQSWVHQIALR (SEQ ID NO: 12), SSSQDGHQWTLFF (SEQ ID NO: 13), MGCDLNSCS (SEQ ID NO: 14), VHQIALRMEVLGCEAQDLY (SEQ ID NO: 15), and KSQYLNNGPQRIGRKYKKVRFM (SEQ ID NO: 16),
optionally wherein at most three, preferably at most two, more preferably at most one amino acid is independently substituted by any other amino acid.

Embodiment 3. The compound of embodiment 2, wherein said amino-acid sequence is selected from SSSQDGHQWTLFF (SEQ ID NO: 13), VHQIALRMEVLGCEAQDLY (SEQ ID NO: 15), and KSQYLNNGPQRIGRKYKKVRFM (SEQ ID NO: 16).

Embodiment 4. The compound of any one of embodiments 1 to 3, wherein the biopolymer scaffold is a human protein.

Embodiment 5. The compound of any one of embodiments 1 to 4, wherein the at least two peptides comprise a peptide P₁ and a peptide P₂, wherein P₁ and P₂ independently comprise a 6-amino-acid fragment as defined in embodiment 1 or 2,
preferably a 7-, more preferably an 8-, more preferably a 9-, even more preferably a 10-, yet even more preferably an 11-, especially a 12-, most preferably a 13-amino-acid fragment as defined in embodiment 3,
wherein P₁ and P₂ are present in form of a peptide dimer P₁-S-P₂, wherein S is a non-peptide spacer, wherein the peptide dimer is covalently bound to the biopolymer scaffold, preferably via a linker.

Embodiment 6. The compound of any one of embodiments 1 to 5, wherein the biopolymer scaffold is selected from human globulins and human albumin.

Embodiment 7. The compound of any one of embodiments 1 to 6, wherein at least one of the at least two peptides, preferably each of the at least two peptides, is circularized. Embodiment 8. The compound of any one of embodiments 1 to 7, wherein the compound is non-immunogenic in humans.

Embodiment 9. The compound of any one of embodiments 1 to 8, wherein each of the peptides independently comprises an amino-acid sequence selected from SEQ ID NOs: 17 to 126, optionally wherein at most three, preferably at most two, more preferably at most one amino acid is independently substituted by any other amino acid, or a 6-, preferably a 7-, more preferably an 8-, more preferably a 9-, even more preferably a 10- yet even more preferably an 11-, most preferably a 12-amino-acid fragment thereof.

Embodiment 10. The compound of any one of embodiments 1 to 9, wherein each of the peptides independently consists an amino-acid sequence selected from SEQ ID NOs: 17 to 126, optionally wherein at most three, preferably at most two, more preferably at most one amino acid is independently substituted by any other amino acid, optionally with an N-terminal and/or C-terminal cysteine residue. Embodiment 11. The compound of any one of embodiments 1 to 10, wherein the biopolymer scaffold is selected from human transferrin and human albumin.

Embodiment 12. A compound, preferably the compound of any one of embodiments 1 to 11, comprising
a biopolymer scaffold and at least
a first peptide n-mer of the general formula:

P (-S-P)₍ₙ₋₁₎ and
a second peptide n-mer of the general formula:

P (-S-P)₍ₙ₋₁₎;

wherein, independently for each occurrence, P is a peptide as defined in any one of embodiments 1 to 10, and S is a non-peptide spacer,
wherein, independently for each of the peptide n-mers, n is an integer of at least 1, preferably of at least 2, more preferably of at least 3, especially of at least 4,
wherein each of the peptide n-mers is bound to the biopolymer scaffold, preferably via a linker each.

Embodiment 13. The compound of embodiment 12, wherein at least one occurrence of P is a circularized peptide, preferably wherein at least 10% of all occurrences of P are circularized peptides, more preferably wherein at least 25% of all occurrences of P are circularized peptides, yet more preferably wherein at least 50% of all occurrences of P are circularized peptides, even more preferably wherein at least 75% of all occurrences of P are circularized peptides, yet even more preferably wherein at least 90% of all occurrences of P are circularized peptides or even wherein at least 95% of all occurrences of P are circularized peptides, especially wherein all of the occurrences of P are circularized peptides.

Embodiment 14. The compound of embodiment 12 or 13, wherein, independently for each of the peptide n-mers, n is at least 2, more preferably at least 3, especially at least 4.

Embodiment 15. The compound of any one of embodiments 12 to 14, wherein, independently for each of the peptide n-mers, n is less than 10, preferably less than 9, more preferably less than 8, even more preferably less than 7, yet even more preferably less than 6, especially less than 5.

Embodiment 16. The compound of any one of embodiments 12 to 15, wherein, for each of the peptide n-mers, n is 2.

Embodiment 17. The compound of any one of embodiments 12 to 16, wherein at least one occurrence of P is Pₐ and/or at least one occurrence of P is P_{b},
wherein Pₐ and P_{b} each independently is a peptide as defined in any one of embodiments 1 to 10.

Embodiment 18. The compound of any one of embodiments 12 to 17, wherein, independently for each occurrence, P is Pₐ or P_{b}.

Embodiment 19. The compound of any one of embodiments 12 to 18, wherein, in the first peptide n-mer, each occurrence of P is Pₐ and, in the second peptide n-mer, each occurrence of P is P_{b}.

Embodiment 20. The compound of any one of embodiments 12 to 19, wherein
the first peptide n-mer is Pa-S-Pₐ and the second peptide n-mer is Pₐ-S-Pₐ; or
the first peptide n-mer is Pa-S-Pₐ and the second peptide n-mer is P_{b}-S-P_{b};
the first peptide n-mer is P_{b} -S-P_{b} and the second peptide n-mer is P_{b}-S-P_{b};
the first peptide n-mer is Pa-S-P_{b} and the second peptide n-mer is Pₐ-S-P_{b};

the first peptide n-mer is Pa-S-P_{b} and the second peptide n-mer is Pₐ-S-Pₐ; or
the first peptide n-mer is Pa-S-P_{b} and the second peptide n-mer is P_{b}-S-P_{b}.

Embodiment 21. A compound comprising
a biopolymer scaffold and at least
a first peptide n-mer which is a peptide dimer of the formula Pₐ-S-Pₐ or Pₐ-S-P_{b},
wherein Pₐ and P_{b} each independently is a peptide as defined in any one of embodiments 1 to 10, and S is a non-peptide spacer,
wherein the first peptide n-mer is bound to the biopolymer scaffold, preferably via a linker. Embodiment 22. The compound of embodiment 21, further comprising a second peptide n-mer which is a peptide dimer of the formula P_{b}-S-P_{b} or Pₐ-S-P_{b},
wherein the second peptide n-mer is bound to the biopolymer scaffold, preferably via a linker.

Embodiment 23. The compound of any one of embodiments 12 to 20 and 22, wherein the first peptide n-mer is different from the second peptide n-mer.

Embodiment 24. The compound of any one of embodiments 17 to 23, wherein the peptide Pₐ is different from the peptide P_{b}, preferably wherein the peptide Pₐ and the peptide P_{b} are two different epitopes of the same antigen or two different epitope parts of the same epitope.

Embodiment 25. The compound of any one of embodiments 17 to 24, wherein the peptide Pₐ and the peptide P_{b} comprise the same amino-acid sequence fragment, wherein the amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.

Embodiment 26. The compound of any one of embodiments 17 to 25, wherein Pₐ and/or P_{b} is circularized.

Embodiment 27. The compound of any one of embodiments 12 to 26, wherein the compound comprises a plurality of said first peptide n-mer and/or a plurality of said second peptide n-mer.

Embodiment 28. The compound of any one of embodiments 1 to 27, wherein the biopolymer scaffold is a protein, preferably a mammalian protein such as a human protein, a non-human primate protein, a sheep protein, a pig protein, a dog protein or a rodent protein.

Embodiment 29. The compound of any one of embodiments 1 to 28, wherein the biopolymer scaffold is a globulin.

Embodiment 30. The compound of any one of embodiments 1 to 29, wherein the biopolymer scaffold is selected from the group consisting of immunoglobulins, alphal-globulins, alpha2-globulins and beta-globulins.

Embodiment 31. The compound of any one of embodiments 1 to 30, wherein the biopolymer scaffold is selected from the group consisting of immunoglobulin G, haptoglobin and transferrin.

Embodiment 32. The compound of any one of embodiments 1 to 31, wherein the biopolymer scaffold is haptoglobin.

Embodiment 33. The compound of any one of embodiments 1 to 28, wherein the biopolymer scaffold is an albumin.

Embodiment 34. The compound of any one of embodiments 1 to 31, wherein the biopolymer scaffold is an anti-CD163 antibody (i.e. an antibody specific for a CD163 protein) or CD163-binding fragment thereof.

Embodiment 35. The compound of embodiment 34, wherein the anti-CD163 antibody or CD163-binding fragment thereof is specific for human CD163 and/or is specific for the extracellular region of CD163, preferably for an SRCR domain of CD163, more preferably for any one of SRCR domains 1-9 of CD163, even more preferably for any one of SRCR domains 1-3 of CD163, especially for SRCR domain 1 of CD163.

Embodiment 36. The compound of embodiment 34, wherein the anti-CD163 antibody or CD163-binding fragment thereof is specific for one of the following peptides:
a peptide consisting of 7-25, preferably 8-20, even more preferably 9-15, especially 10-13 amino acids, wherein the peptide comprises the amino acid sequence CSGRVEVKVQEEWGTVCNNGWSMEA (SEQ ID NO: 139) or a 7-24 amino-acid fragment thereof,
a peptide consisting of 7-25, preferably 8-20, even more preferably 9-15, especially 10-13 amino acids, wherein the peptide comprises the amino acid sequence DHVSCRGNESALWDCKHDGWG (SEQ ID NO: 149) or a 7-20 amino-acid fragment thereof, or
a peptide consisting of 7-25, preferably 8-20, even more preferably 9-15, especially 10-13 amino acids, wherein the peptide comprises the amino acid sequence SSLGGTDKELRLVDGENKCS (SEQ ID NO: 160) or a 7-19 amino-acid fragment thereof.

Embodiment 37. The compound of embodiment 36, wherein the anti-CD163 antibody or CD163-bindinq fragment thereof is specific for a peptide comprising the amino acid sequence ESALW (SEQ ID NO: 150) or ALW.

Embodiment 38. The compound of embodiment 36, wherein the anti-CD163 antibody or CD163-binding fragment thereof is specific for a peptide comprising the amino acid sequence CRVEVKVQWEEW (SEQ ID NO: 140), WGTVCNNGWS (SEQ ID NO: 141) or WCTVCNNGW (SEQ ID NO: 149).

Embodiment 39. The compound of embodiment 36, wherein the anti-CD163 antibody or CD163-bindinq fragment thereof is specific for a peptide comprising the amino acid sequence SSLGCTDKELR (SEQ ID NO: 161) or SSLGG (SEQ ID NO: 162).

Embodiment 40. The compound of any one of embodiments 1 to 39, wherein the compound is non-immunogenic in a mammal, preferably in a human, in a non-human primate, in a sheep, in a pig, in a dog or in a rodent.

Embodiment 41. The compound of any one of embodiments 1 to 40, wherein the compound is for intracorporeal sequestration (or intracorporeal depletion) of at least one antibody in an individual, preferably in the bloodstream of the individual and/or for reduction of the titre of at least one antibody in the individual, preferably in the bloodstream of the individual.

Embodiment 42. The compound of any one embodiments 1 to 41, wherein the compound further comprises at least
a third peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is P_{c}, wherein P_{c} is a peptide as defined in any one of embodiments 1 to 10,
preferably wherein P_{c} is circularized.

Embodiment 43. The compound of embodiment 42, wherein the compound further comprises at least
a fourth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is P_{d}, wherein P_{d} is a peptide as defined in any one of embodiments 1 to 10,
preferably wherein P_{d} is circularized; Embodiment 44. The compound of embodiment 43, wherein the compound further comprises at least
a fifth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is Pₑ, wherein Pₑ is a peptide as defined in any one of embodiments 1 to 10,
preferably wherein Pe is circularized; Embodiment 45. The compound of embodiment 44, wherein the compound further comprises at least a sixth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is P_{f}, wherein P_{f} is a peptide as defined in any one of embodiments 1 to 10,
preferably wherein Pf is circularized; Embodiment 46. The compound of embodiment 45, wherein the compound further comprises at least a seventh peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is P_{g}, wherein P_{g} is a peptide as defined in any one of embodiments 1 to 10,
preferably wherein Pg is circularized;

Embodiment 47. The compound of embodiment 46, wherein the compound further comprises at least
an eigth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is Pₕ, wherein Pₕ is a peptide as defined in any one of embodiments 1 to 10,
preferably wherein Ph is circularized;

Embodiment 48. The compound of embodiment 47, wherein the compound further comprises at least
a ninth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is Pᵢ, wherein Pᵢ is a peptide as defined in any one of embodiments 1 to 10,
preferably wherein Pᵢ is circularized;

Embodiment 49. The compound of embodiment 48, wherein the compound further comprises at least
a tenth peptide n-mer of the general formula:

P (-S-P)₍ₙ₋₁₎,
wherein, independently for each occurrence, P is a peptide as defined in any one of embodiments 1 to 10, and S is a non-peptide spacer,
preferably wherein each occurrence of P is Pⱼ, wherein Pⱼ is a peptide as defined in any one of embodiments 1 to 10,
preferably wherein Pⱼ is circularized.

Embodiment 50. The compound of any one of embodiments 12 to 49, wherein each of the peptide n-mers is covalently bound to the biopolymer scaffold, preferably via a linker each. Embodiment 51. The compound of any one of embodiments 5 to 50, wherein at least one of said linkers is selected from disulphide bridges and PEG molecules.

Embodiment 52. The compound of any one of embodiments 5 to 51, wherein at least one of the spacers S is selected from PEG molecules or glycans.

Embodiment 53. The compound of any one of embodiments 17 to 52, wherein the first peptide n-mer is Pₐ-S-P_{b} and the second peptide n-mer is Pₐ-S-P_{b}.

Embodiment 54. The compound of any one of embodiments 17 to 53, wherein the peptide Pₐ and the peptide P_{b} comprise the same amino-acid sequence fragment, wherein the amino-acid sequence fragment has a length of at least 5 amino acids, even more preferably at least 6 amino acids, yet even more preferably at least 7 amino acids, especially at least 8 amino acids or even at least 9 amino acids.

Embodiment 55. The compound of any one of embodiments 1 to 54, wherein the compounds is for the sequestration (or depletion) of anti-(human) factor VIII antibodies.

Embodiment 56. A pharmaceutical composition comprising the compound of any one of embodiments 1 to 55 and at least one pharmaceutically acceptable excipient.

Embodiment 57. The pharmaceutical composition of embodiment 56, wherein the molar ratio of the peptides to scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 58. The pharmaceutical composition of embodiment 56 or 57, wherein the composition is prepared for intraperitoneal, subcutaneous, intramuscular and/or intravenous administration and/or wherein the composition is for repeated administration.

Embodiment 59. The pharmaceutical composition of any one of embodiments 56 to 58, or the compound of any one of embodiments 5 to 55, wherein the molar ratio of peptide P to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 60. The pharmaceutical composition of any one of embodiments 56 to 59, or the compound of any one of embodiments 17 to 55 wherein the molar ratio of peptide Pₐ to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 61. The pharmaceutical composition of any one of embodiments 56 to 60, or the compound of any one of embodiments 17 to 55, wherein the molar ratio of peptide P_{b} to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

Embodiment 62. The pharmaceutical composition of any one of embodiments 56 to 61, further comprising a factor VIII replacement product, preferably factor VIII, most preferably human factor VIII.

Embodiment 63. The pharmaceutical composition of any one of embodiments 56 to 62 for use in therapy.

Embodiment 64. The pharmaceutical composition of any one of embodiments 56 to 62 for use in prevention or treatment of hemophilia A in an individual.

Embodiment 65. The pharmaceutical composition for use of embodiment 64, wherein the hemophilia A is congenital hemophilia A and/or acquired hemophilia A.

Embodiment 66. The pharmaceutical composition for use of any one of embodiments 63 to 65, wherein the pharmaceutical composition is administered to the individual, and wherein a factor VIII replacement product, preferably factor VIII, most preferably human factor VIII, is administered to the individual in combination with said composition, before said composition is administered, or after said composition has been administered, preferably wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window, especially wherein this window is followed by administration of the factor VIII replacement product, preferably factor VIII, most preferably human factor VIII, within 24 hours, preferably within 12 hours.

Embodiment 67. The pharmaceutical composition of any one of embodiments 56 to 61 for use in inhibiting neutralization and/or inhibition of a factor VIII replacement product, preferably factor VIII, more preferably human factor VIII, in an individual, preferably wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window, especially wherein this window is followed by administration of the factor VIII replacement product within 24 hours, preferably within 12 hours.

Embodiment 68. The pharmaceutical composition for use according to any one of embodiments 63 to 67, wherein the composition is administered at a dose of 1-1000 mg, preferably 2-500 mg, more preferably 3-250 mg, even more preferably 4-100 mg, especially 5-50 mg, compound per kg body weight of the individual.

Embodiment 69. The pharmaceutical composition for use according to any one of embodiments 63 to 68, wherein the composition is administered intraperitoneally, subcutaneously, intramuscularly or intravenously.

Embodiment 70. The pharmaceutical composition for use according to any one of embodiments 63 to 69, wherein one or more antibodies are present in the individual which are specific for at least one occurrence of peptide P, or for peptide Pₐ and/or peptide P_{b}, preferably wherein said antibodies are specific for factor VIII, preferably human factor VIII.

Embodiment 71. The pharmaceutical composition for use according to any one of embodiments 63 to 70, wherein one or more factor VIII neutralizing or inhibiting antibodies are present in the individual.

Embodiment 72. The pharmaceutical composition for use according to any one of embodiments 63 to 71, wherein the composition is non-immunogenic in the individual.

Embodiment 73. The pharmaceutical composition for use according to any one of embodiments 63 to 72, wherein the composition is administered at a dose of 1-1000 mg, preferably 2-500 mg, more preferably 3-250 mg, even more preferably 4-100 mg, especially 5-50 mg, compound per kg body weight of the individual.

Embodiment 74. The pharmaceutical composition for use according to any one of embodiments 63 to 73, wherein the individual further receives factor VIII replacement therapy, preferably wherein the individual is administered a factor VIII replacement product, preferably factor VIII, most preferably human factor VIII, preferably wherein said administering of the factor VIII replacement product is prior to, concurrent with and/or subsequent to administering of the pharmaceutical composition.

Embodiment 75. A method of ameliorating or treating hemophilia A in an individual in need thereof, comprising
obtaining a pharmaceutical composition as defined in any one of embodiments 56 to 62; and
administering an effective amount of the pharmaceutical composition to the individual.

Embodiment 76. The method according to embodiment 75, wherein the method is defined as in any one of embodiments 63 to 74.

Embodiment 77. A method of sequestering (or depleting) one or more antibodies present in an individual, comprising
obtaining a pharmaceutical composition as defined in any one of embodiments 56 to 62, wherein the composition is non-immunogenic in the individual and wherein the one or more antibodies present in the individual are specific for at least one occurrence of P, or for peptide Pₐ and/or peptide P_{b}; and
administering the pharmaceutical composition to the individual.

Embodiment 78. The method of embodiment 77, wherein the one or more antibodies are specific for factor VIII, preferably human factor VIII.

Embodiment 79. The method of embodiment 77 or 78, wherein the individual is a non-human animal, preferably a non-human primate, a sheep, a pig, a dog or a rodent, in particular a mouse.

Embodiment 80. The method of any one of embodiments 77 to 79, wherein the biopolymer scaffold is autologous with respect to the individual, preferably wherein the biopolymer scaffold is an autologous protein.

Embodiment 81. The method of any one of embodiments 77 to 80, wherein the individual is administered a factor VIII replacement product, and wherein the one or more antibodies present in the individual are specific for said factor VIII replacement product, preferably wherein said administering of the factor VIII replacement product is prior to, concurrent with and/or subsequent to said administering of the pharmaceutical composition,
preferably wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window, especially wherein this window is followed by administration of the factor VIII replacement product within 24 hours, preferably within 12 hours.

Embodiment 82. The method of embodiment 81, wherein the factor VIII replacement product is factor VIII, preferably human factor VIII.

Embodiment 83. The method of any one of embodiments 77 to 82, wherein the composition is administered intraperitoneally, subcutaneously, intramuscularly or intravenously.

Embodiment 84. A peptide, wherein the peptide is defined as in any one of embodiments 1 to 10.

Embodiment 85. A method for detecting and/or quantifying anti-factor VIII antibodies in a biological sample comprising the steps of
bringing the sample into contact with the peptide of embodiment 84, and
detecting the presence and/or concentration of anti-factor VIII antibodies in the sample.

Embodiment 86. The method of embodiment 85, wherein the peptide is immobilized on a solid support, in particular a biosensor-based diagnostic device with an electrochemical, fluorescent, magnetic, electronic, gravimetric or optical biotransducer and/or wherein the peptide is coupled to a reporter or reporter fragment, such as a reporter fragment suitable for a PCA.

Embodiment 87. The method of embodiment 85 or 86, wherein the method is a sandwich assay, preferably an enzyme-linked immunosorbent assay (ELISA).

Embodiment 88. The method of any one of embodiments 85 to 87, wherein the sample is obtained from a mammal, preferably a human.

Embodiment 89. The method of any one of embodiment 85 to 88, wherein the sample is a blood sample, preferably whole blood, serum, or plasma.

Embodiment 90. Use of the peptide according to embodiment 84 in an enzyme-linked immunosorbent assay (ELISA), preferably for a method as defined in any one of embodiments 85 to 89.

Embodiment 91. Diagnostic device comprising the peptide according to embodiment 84, wherein the peptide is immobilized on a solid support and/or wherein the peptide is coupled to a reporter or reporter fragment, such as a reporter fragment suitable for a PCA

Embodiment 92. Diagnostic device according to embodiment 91, wherein the solid support is an ELISA plate or a surface plasmon resonance chip.

Embodiment 93. Diagnostic device according to embodiment 91, wherein the diagnostic device is a lateral flow assay device or a biosensor-based diagnostic device with an electrochemical, fluorescent, magnetic, electronic, gravimetric or optical biotransducer.

Embodiment 94. A diagnostic kit comprising a peptide according to embodiment 84, preferably diagnostic device according to any one of embodiment 91 to 93, and preferably one or more selected from the group of a buffer, a reagent, instructions.

Embodiment 95. An apheresis device comprising the peptide according to embodiment 84, preferably immobilized on a solid carrier.

Embodiment 96. The apheresis device according to embodiment 95, wherein the solid carrier is capable of being contacted with blood or plasma flow.

Embodiment 97. The apheresis device according to embodiment 95 or 96, wherein the solid carrier comprises the compound according to any one of embodiments 1 to 55.

Embodiment 98. The apheresis device according to any one of embodiment 95 to 97, wherein the solid carrier is a sterile and pyrogen-free column.

Embodiment 99. The apheresis device according to any one of embodiments 95 to 98, wherein the apheresis device comprises at least two, preferably at least three, more preferably at least four different peptides according to embodiment 84.

The present invention is further illustrated by the following figures and examples, without being restricted thereto.

In the context of the following figures and examples the compound on which the inventive approach is based is also referred to as “Selective Antibody Depletion Compound” (SADC).

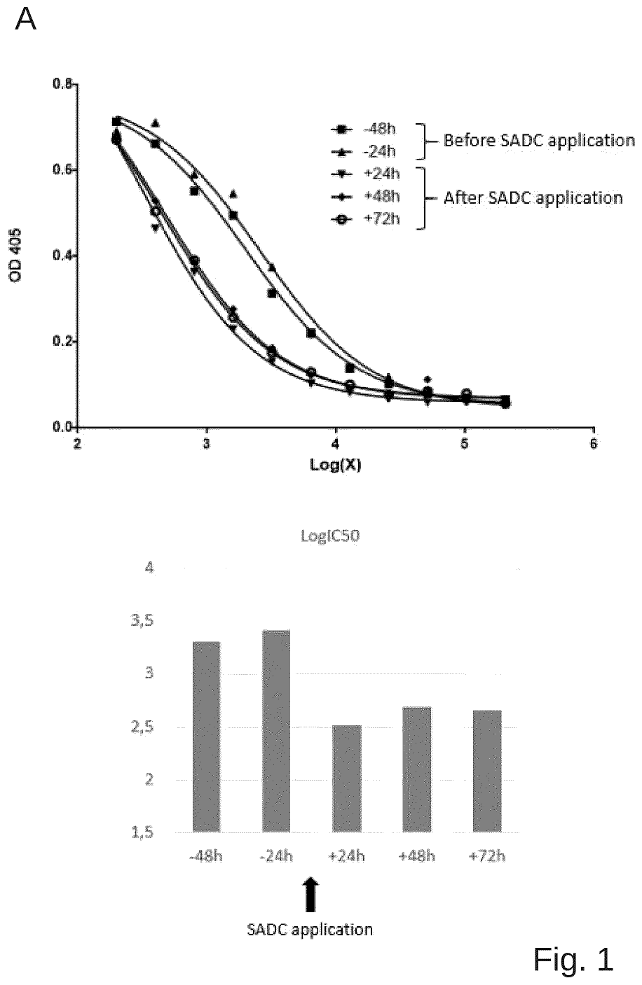

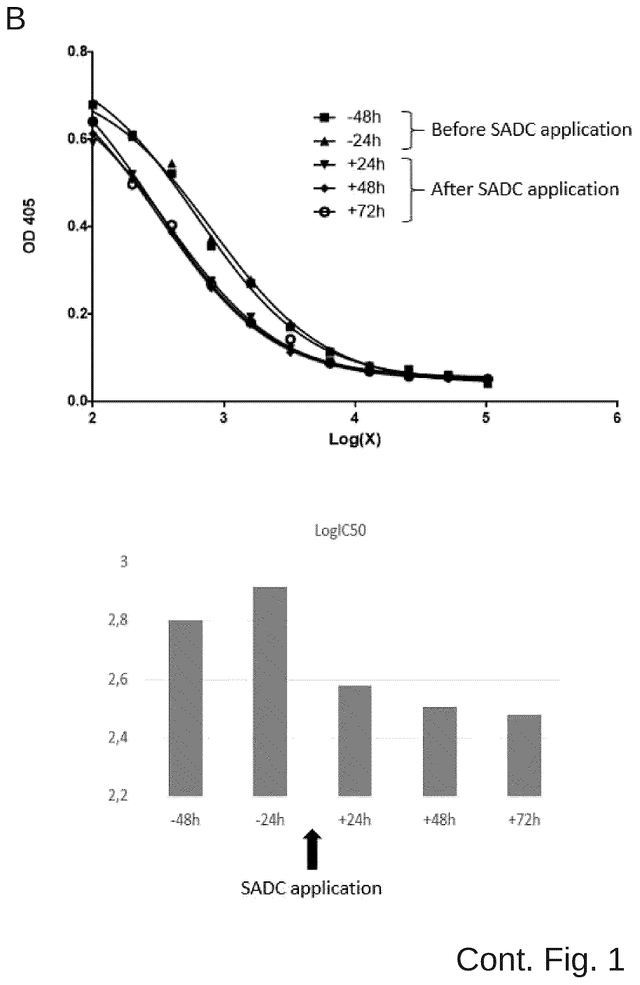

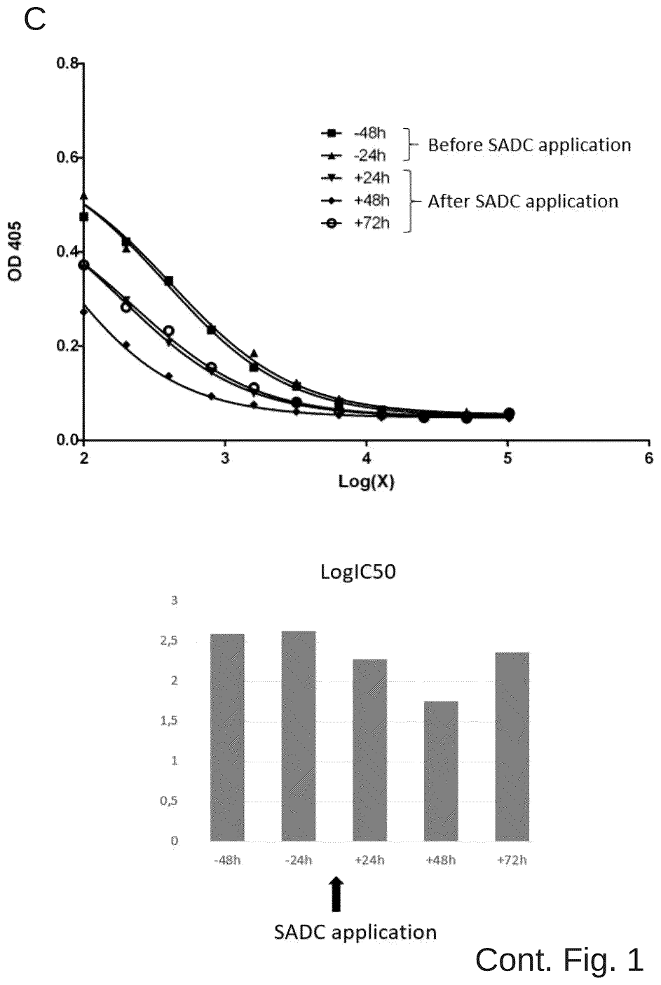

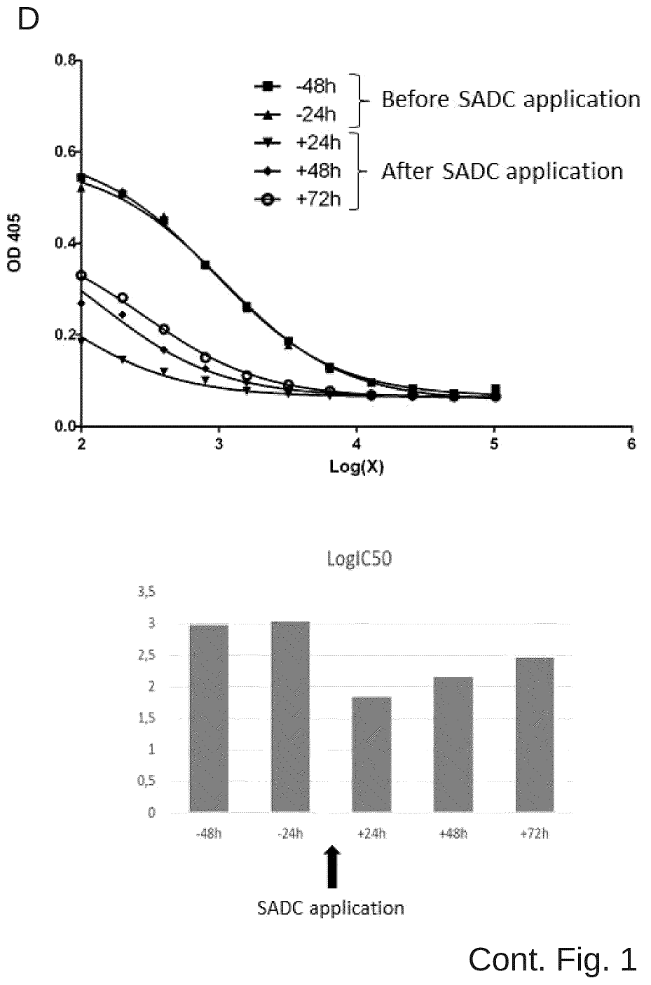

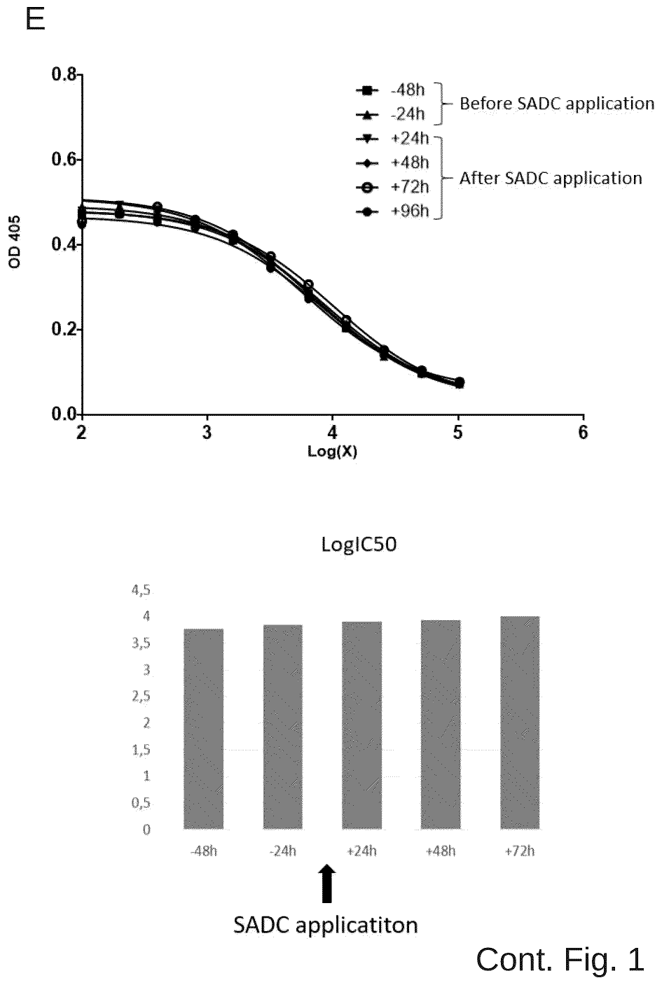

FIG. 1: SADCs successfully reduce the titre of undesired antibodies. Each compound was applied at time point 0 by i.p. injection into Balb/c mice pre-immunized by peptide immunization against a defined antigen. Each top panel shows anti-peptide titers (0.5x dilution steps; X-axis shows log(X) dilutions) against OD values (y-axis) according to a standard ELISA detecting the corresponding antibody. Each bottom panel shows titers LogIC50 (y-axis) before injection of each compound of the invention (i.e. titers at -48 h and -24 h) and after application of each compound of the invention (i.e. titers+24 h,+48 h and +72 h after injection; indicated on the x-axis). (A) Compound with albumin as the biopolymer scaffold that binds to antibodies directed against EBNA1 (associated with pre-eclampsia). The mice were pre-immunized with a peptide vaccine carrying the EBNA-1 model epitope. (B) Compound with albumin as the biopolymer scaffold that binds to antibodies directed against a peptide derived from the human AChR protein MIR (associated with myasthenia gravis). The mice were pre-immunized with a peptide vaccine carrying the AChR MIR model epitope. (C) Compound with immunoglobulin as the biopolymer scaffold that binds to antibodies directed against EBNA1 (associated with pre-eclampsia). The mice were pre-immunized with a peptide vaccine carrying the EBNA-1 model epitope. (D) Compound with haptoglobin as the biopolymer scaffold that binds to antibodies directed against EBNA1 (associated with pre-eclampsia). The mice were pre-immunized with a peptide vaccine carrying the EBNA-1 model epitope. (E) Demonstration of selectivity using the same immunoglobulin-based compound of the invention binding to antibodies directed against EBNA1 that was used in the experiment shown in panel C. The mice were pre-immunized with an unrelated amino acid sequence. No titre reduction occurred, demonstrating selectivity of the compound.

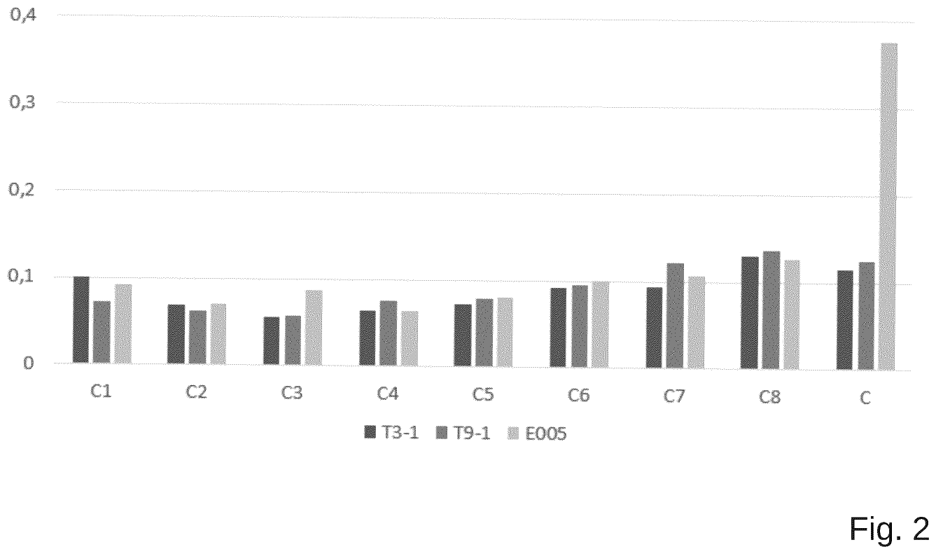

FIG. 2: SADCs are non-immunogenic and do not induce antibody formation after repeated injection into mice. Animals C1-C4 as well as animals C5-C8 were treated i.p. with two different compounds of the invention. Control animal C was vaccinated with a KLH-peptide derived from the human AChR protein MIR. Using BSA-conjugated peptide probes T3-1, T9-1 and E005 (grey bars, as indicated in the graph), respectively, for antibody titer detection by standard ELISA at a dilution of 1:100, it could be demonstrated that antibody induction was absent in animals treated with a compound of the invention, when compared to the vaccine-treated control animal C (y-axis, OD450 nm).

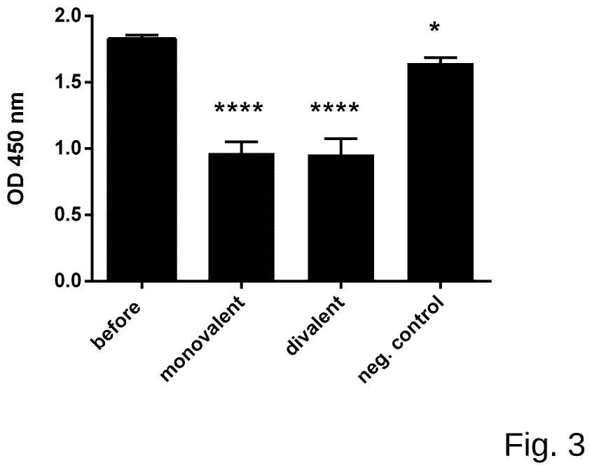

FIG. 3: Successful in vitro depletion of antibodies using SADCs carrying multiple copies of monovalent or divalent peptides. SADCs with mono- or divalent peptides were very suitable to adsorb antibodies and thereby deplete them. “Monovalent” means that peptide monomers are bound to the biopolymer scaffold (i.e. n=1) whereas “divalent” means that peptide dimers are bound to the biopolymer scaffold (i.e. n=2). In the present case, the divalent peptides were “homodivalent”, i.e. the peptide n-mer of the SADC is E006-spacer-E006).

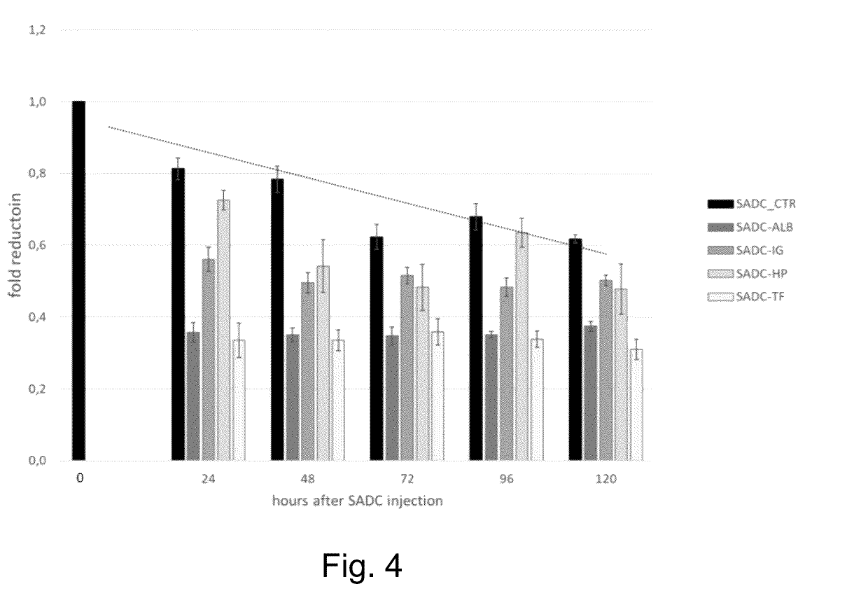

FIG. 4: Rapid, selective antibody depletion in mice using various SADC biopolymer scaffolds. Treated groups exhibited rapid and pronounced antibody reduction already at 24 hrs (in particular SADC-TF) when compared to the mock treated control group SADC-CTL (containing an unrelated peptide). SADC with albumin scaffold-SADC-ALB, SADC with immunoglobulin scaffold-SADC-IG, SADC with haptoglobin scaffold - SADC-HP, and SADC with transferrin scaffold-SADC-TF.

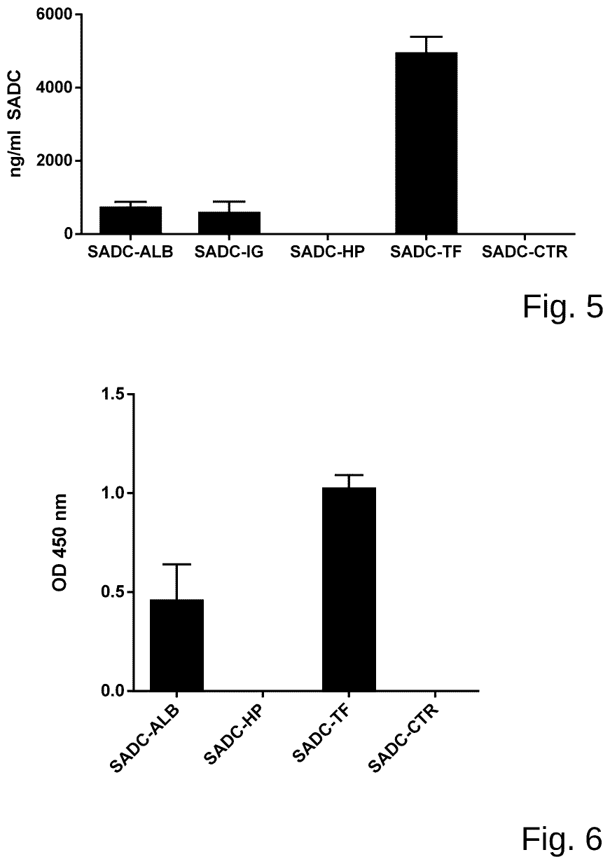

FIG. 5: Detection of SADCs in plasma via their peptide moieties 24 hrs after SADC injection. Both haptoglobin-scaffold-based SADCs (SADC-HP and SADC-CTL) exhibited a relatively shorter plasma half life which represents an advantage over SADCs with other biopolymer scaffolds such as SADC-ALB, SADC-IG oder SADC-TF. SADC with albumin scaffold-SADC-ALB, SADC with immunoglobulin scaffold-SADC-IG, SADC with haptoglobin scaffold-SADC-HP, and SADC with transferrin scaffold-SADC-TF.

FIG. 6: Detection of SADC-IgG complexes in plasma 24 hrs after SADC injection. Haptoglobin based SADCs were subject to accelerated clearance when compared to SADCs with other biopolymer scaffolds. SADC with albumin scaffold-SADC-ALB, SADC with immunoglobulin scaffold-SADC-IG, SADC with haptoglobin scaffold-SADC-HP, and SADC with transferrin scaffold-SADC-TF.

FIG. 7: In vitro analysis of SADC-IgG complex formation. Animals SADC-TF and -ALB showed pronounced immunocomplex formation and binding to Clq as reflected by the strong signals and by sharp signal lowering in case 1000ng/ml SADC-TF due to the transition from antigen-antibody equilibrium to antigen excess. In contrast, in vitro immunocomplex formation with SADC-HP or SADC-IG were much less efficient when measured in the present assay. These findings corroborate the finding that haptoglobin scaffolds are advantageous over other SADC biopolymer scaffolds because of the reduced propensity to activate the complement system. SADC with albumin scaffold-SADC-ALB, SADC with immunoglobulin scaffold-SADC-IG, SADC with haptoglobin scaffold-SADC-HP, and SADC with transferrin scaffold-SADC-TF.

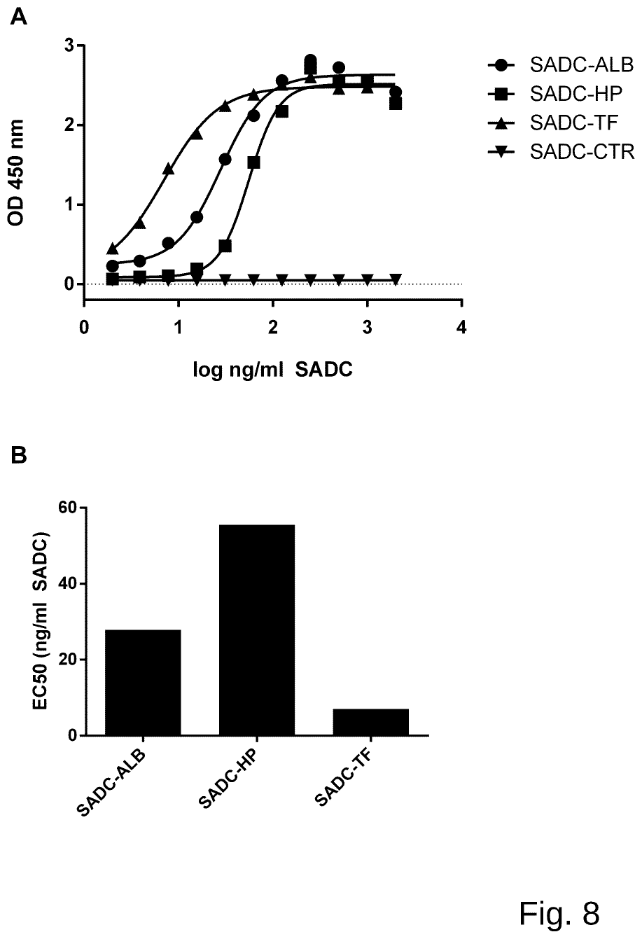

FIG. 8: Determination of IgG capturing by SADCs in vitro. SADC-HP showed markedly less antibody binding capacity in vitro when compared to SADC-TF or SADC-ALB. SADC with albumin scaffold-SADC-ALB, SADC with immunoglobulin scaffold-SADC-IG, SADC with haptoglobin scaffold -SADC-HP, and SADC with transferrin scaffold-SADC-TF.

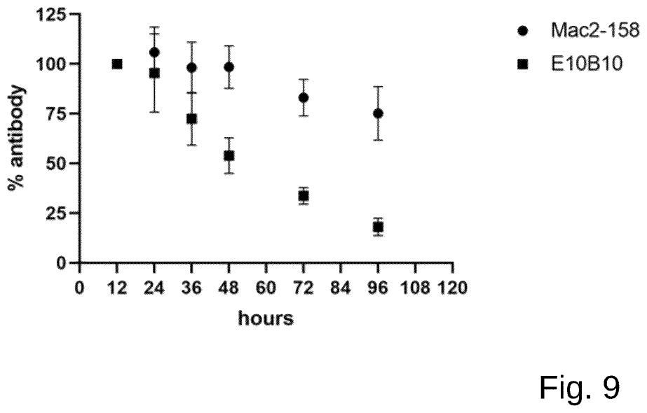

FIG. 9: Blood clearance of an anti-CD163-antibody-based biopolymer scaffold. In a mouse model, mAb E10B10 (specific for murine CD163) is much more rapidly cleared from circulation than mAb Mac2-158 (specific for human CD163 but not for murine CD163, thus serving as negative control in this experiment).

### EXAMPLES

Examples 1-10 relate to the general working principle of SADCs, demonstrating the selective removal of antibodies. Example 11-12 relate to the specific application of this therapeutic concept to factor VIII and hemophilia A.

### Example 1: SADCs Effectively Reduce the Titre of Undesired Antibodies

Animal models: In order to provide in vivo models with measurable titers of prototypic undesired antibodies in human indications, BALB/c mice were immunized using standard experimental vaccination with KLH-conjugated peptide vaccines derived from established human autoantigens or anti-drug antibodies. After titer evaluation by standard peptide ELISA, immunized animals were treated with the corresponding test SADCs to demonstrate selective antibody lowering by SADC treatment. All experiments were performed in compliance with the guidelines by the corresponding animal ethics authorities.

Immunization of mice with model antigens: Female BALB/c mice (aged 8-10 weeks) were supplied by Janvier (France), maintained under a 12 h light/12 h dark cycle and given free access to food and water. Immunizations were performed by s.c. application of KLH carrier-conjugated peptide vaccines injected 3 times in biweekly intervals. KLH conjugates were generated with peptide T3-2 (SEQ ID NO. 127: CGRPQKRPSCIGCKG), which represents an example for molecular mimicry between a viral antigen (EBNA-1) and an endogenous human receptor antigen, namely the placental GPR50 protein, that was shown to be relevant to preeclampsia (Elliott et al.). In order to confirm the generality of this approach, a larger antigenic peptide derived from the autoimmune condition myasthenia gravis was used for immunization of mice with a human autoepitope. In analogy to peptide T3-2, animals were immunized with peptide T1-1 (SEQ ID NO. 128: LKWNPDDYGGVKKIHIPSEKGC), derived from the MIR (main immunogenic region) of the human AChR protein which plays a fundamental role in pathogenesis of the disease (Luo et al.). The T1-1 peptide was used for immunizing mice with a surrogate partial model epitope of the human AChR autoantigen. The peptide T8-1 (SEQ ID NO. 129: DHTLYTPYHTHPG) was used to immunize control mice to provide a control titer for proof of selectivity of the system. For vaccine conjugate preparation, KLH carrier (Sigma) was activated with sulfo-GMBS (Cat. Nr. 22324 Thermo), according to the manufacturer's instructions, followed by addition of either N- or C-terminally cysteinylated peptides T3-2 and T1-1 and final addition of Alhydrogel° before injection into the flank of the animals. The doses for vaccines T3-2 and T1-1 were 15 μg of conjugate in a volume of 100u1 per injection containing Alhydrogel° (InvivoGen VAC-Alu-250) at a final concentration of 1% per dose.

Generation of prototypic SADCs: For testing selective antibody lowering activity by SADCs of T3-2 and T1-1 immunized mice, SADCs were prepared with mouse serum albumin (MSA) or mouse immunoglobulin (mouse-Ig) as biopolymer scaffold in order to provide an autologous biopolymer scaffold, that will not induce any immune reaction in mice, or non-autologuous human haptoglobin as biopolymer scaffold (that did not induce an allogenic reaction after one-time injection within 72 hours). N-terminally cysteinylated SADC peptide E049 (SEQ ID NO. 130: GRPQKRPSCIG) and/or C-terminally cysteinylated SADC peptide E006 (SEQ ID NO. 131: VKKIHIPSEKG) were linked to the scaffold using sulfo-GMBS (Cat. Nr. 22324 Thermo)-activated MSA (Sigma; Cat. Nr. A3559) or -mouse-Ig (Sigma, 15381) or -human haptoglobin (Sigma H0138) according to the instructions of the manufacturer, thereby providing MSA-, Ig- and haptoglobin-based SADCs with the corresponding cysteinylated peptides, that were covalently attached to the lysines of the corresponding biopolymer scaffold. Beside conjugation of the cysteinylated peptides to the lysines via a bifunctional amine-to-sulfhydryl crosslinker, a portion of the added cysteinylated SADC peptides directly reacted with sulfhydryl groups of cysteins of the albumin scaffold protein, which can be detected by treating the conjugates with DTT followed by subsequent detection of free peptides using mass spectrometry or any other analytical method that detects free peptide. Finally, these SADC conjugates were dialysed against water using Pur-A-LyzerTM (Sigma) and subsequently lyophilized. The lyophilized material was resuspended in PBS before injection into animals.

In vivo functional testing of SADCs: Prototypic SADCs, SADC-E049 and SADC-E006 were injected intraperitoneally (i.p.; as a surrogate for an intended intravenous application in humans and larger animals) into the mice that had previously been immunized with peptide vaccine T3-2 (carrying the EBNA-1 model epitope) and peptide vaccine T1-1 (carrying the AChR MIR model epitope). The applied dose was 30 μg SADC conjugate in a volume of 5 0μl PBS. Blood takes were performed by submandibular vein puncture, before (−48 h, −24 h) and after (+24 h,+48 h,+72 h, etc.) i.p. SADC injections, respectively, using capillary micro-hematocrit tubes. Using ELISA analysis (see below), it was found that both prototypic SADCs were able to clearly reduce the titers over a period of at least 72 hrs in the present animal model. It could therefore be concluded that SADCs can be used to effectively reduce titers in vivo.

Titer analysis: Peptide ELISAs were performed according to standard procedures using 96-well plates (Nunc Medisorp plates; Thermofisher, Cat Nr 467320) coated for lh at RT with BSA-coupled peptides (30 nM, dissolved in PBS) and incubated with the appropriate buffers while shaking (blocking buffer, 1% BSA, 1×PBS; washing buffer, 1×PBS/0.1% Tween; dilution buffer, 1×PBS/0.1% BSA/0.1% Tween). After serum incubation (dilutions starting at 1:50 in PBS; typically in 1:3 or 1:2 titration steps), bound antibodies were detected using Horseradish Peroxidase-conjugated goat anti-mouse IgG (Fc) from Jackson immunoresearch (115-035-008). After stopping the reaction, plates were measured at 450nm for 20min using TMB. EC50 were calculated from readout values using curve fitting with a 4-parameter logistic regression model (GraphPad Prism) according to the procedures recommended by the manufacturer. Constraining parameters for ceiling and floor values were set accordingly, providing curve fitting quality levels of R²>0.98.

FIG. 1A shows an in vivo proof of concept in a mouse model for in vivo selective plasma-lowering activity of a prototypic albumin-based SADC candidate that binds to antibodies directed against EBNA1, as a model for autoantibodies and mimicry in preeclampsia (Elliott et al.). For these mouse experiments, mouse albumin was used, in order to avoid any reactivity against a protein from a foreign species. Antibody titers were induced in 6 months old Balb/c mice by standard peptide vaccination. The bottom panel demonstrates that titers LogIC50 (y-axis) before SADC injection (i.e. titers at −48 h and −24 h) were higher than titers LogIC50 after SADC application (i.e. titers+24 h,+48 h and+72 h after injection; indicated on the x-axis).

A similar example is shown in FIG. 1B, using an alternative example of a peptidic antibody binding moiety for a different disease indication. Antibody lowering activity of an albumin-based SADC in a mouse model that was pre-immunized with a different peptide derived from the human AChR protein MIR region (Luo et al.) in order to mimic the situation in myasthenia gravis. The induced antibody titers against the AChR-MIR region were used as surrogate for anti-AChR-MIR autoantibodies known to play a causative role in myasthenia gravis (reviewed by Vincent et al.). A clear titer reduction was seen after SADC application.

FIGS. 1C and 1D demonstrate the functionality of SADC variants comprising alternative biopolymer scaffolds. Specifically, FIG. 1C shows that an immunoglobulin scaffold can be successfully used whereas FIG. 1D demonstrates the use of a haptoglobin-scaffold for constructing an SADC. Both examples show an in vivo proof of concept for selective antibody lowering by an SADC, carrying covalently bound example peptide E049.

The haptoglobin-based SADC was generated using human Haptoglobin as a surrogate although the autologuous scaffold protein would be preferred. In order to avoid formation of anti-human-haptoglobin antibodies, only one single SADC injection per mouse of the non-autologuous scaffold haptoglobin was used for the present experimental conditions. As expected, under the present experimental conditions (i.e. one-time application), no antibody reactivity was observed against the present surrogate haptoglobin homologue.

FIG. 1E demonstrates the selectivity of the SADC system. The immunoglobulin-based SADC carrying the peptide E049 (i.e. the same as in FIG. 1C) cannot reduce the Ig-titer that was induced by a peptide vaccine with an unrelated, irrelevant aminoacid sequence, designated peptide T8-1 (SEQ ID NO. 129: DHTLYTPYHTHPG). The example shows an in vivo proof of concept for the selectivity of the system. The top panel shows anti-peptide T8-1 titers (0,5x dilution steps starting from 1:50 to 1:102400; X-axis shows log(X) dilutions) against OD values (y-axis) according to a standard ELISA. T8-1-titers are unaffected by administration of SADC-Ig-E049 after application. The bottom panel demonstrates that the initial titers LogIC50 (y-axis) before SADC injection (i.e. titers at -48 h and -24 h) are unaffected by administration of SADC-Ig-E049 (arrow) when compared to the titers LogIC50 after SADC application (i.e. titers+24 h,+48 h and+72 h; as indicated on the x-axis), thereby demonstrating the selectivity of the system.

### Example 2: Immunogenicity of SADCs

In order to exclude immunogenicity of SADCs, prototypic candidate SADCs were tested for their propensity to induce antibodies upon repeated injection. Peptides T3-1 and T9-1 were used for this test. T3-1 is a 10-amino acid peptide derived from a reference epitope of the Angiotensin receptor, against which agonistic autoantibodies are formed in a pre-eclampsia animal model (Zhou et al.); T9-1 is a 12-amino acid peptide derived from a reference anti-drug antibody epitope of human IFN gamma (Lin et al.). These control SADC conjugates were injected 8× every two weeks i.p. into naive, non-immunized female BALB/c mice starting at an age of 8-10 weeks.

Animals C1-C4 were treated i.p. (as described in example 1) with SADC T3-1. Animals C5-C8 were treated i.p. with an SADC carrying the peptide T9-1. As a reference signal for ELISA analysis, plasma from a control animal that was vaccinated 3 times with KLH-peptide T1-1 (derived from the AChR-MIR, explained in Example 1) was used. Using BSA-conjugated peptide probes T3-1, T9-1 and E005 (SEQ ID NO. 132: GGVKKIHIPSEK), respectively, for antibody titer detection by standard ELISA at a dilution of 1:100, it could be demonstrated that antibody induction was absent in SADC-treated animals, when compared to the vaccine-treated control animal C (see FIG. 2). The plasmas were obtained by submandibular blood collection, 1 week after the 3rd vaccine injection (control animal C) and after the last of 8 consecutive SADC injections in 2-weeks intervals (animals C1-C8), respectively. Thus it was demonstrated that SADCs are non-immunogenic and do not induce antibody formation after repeated injection into mice.

### Example 3: Successful in Vitro Depletion of Antibodies Using SADCs Carrying Multiple Copies of Monovalent or Divalent Peptides

Plasma of E006-KLH (VKKIHIPSEKG (SEQ ID NO: 131) with C-terminal cysteine, conjugated to KLH) vaccinated mice was diluted 1:3200 in dilution buffer (PBS+0.1% w/v BSA+0.1% Tween20) and incubated (100 μl, room temperature) sequentially (10 min/well) four times on single wells of a microtiter plate that was coated with 2.5 μg/ml (250 ng/well) of SADC or 5 μg/ml (500 ng/well) albumin as negative control.

In order to determine the amount of free, unbound antibody present before and after incubation on SADC coated wells, 50 pl of the diluted serum were taken before and after the depletion and quantified by standard ELISA using E006-BSA coated plates (10 nM peptide) and detection by goat anti mouse IgG bio (Southern Biotech, diluted 1:2000). Subsequently, the biotinylated antibody was detected with Streptavidin-HRP (Thermo Scientific, diluted 1:5000) using TMB as substrate. Development of the signal was stopped with 0.5 M sulfuric acid.

ELISA was measured at OD450 nm (y-axis). As a result, the antibody was efficiently adsorbed by either coated mono- or divalent SADCs containing peptide E006 with C-terminal cysteine (sequence VKKIHIPSEKGC, SEQ ID NO: 133) (before=non-depleted starting material; mono-divalent corresponds to peptides displayed on the SADC surface; neg. control was albumin; indicated on the x-axis). See FIG. 3. (“Monovalent” means that peptide monomers are bound to the biopolymer scaffold (i.e. n=1) whereas “divalent” means that peptide dimers are bound to the biopolymer scaffold (i.e. n=2). In the present case, the divalent peptides were “homodivalent”, i.e. the peptide n-mer of the SADC is E006-S-E006.)

This demonstrates that SADCs with mono- or divalent peptides are very suitable to adsorb antibodies and thereby deplete them.

### Example 4: Generation of Mimotope-Based SADCs

Linear and circular peptides derived from wild-type or modified peptide amino acid sequences can be used for the construction of specific SADCs for the selective removal of harmful, disease-causing or otherwise unwanted antibodies directed against a particular epitope. In case of a particular epitope, linear peptides or constrained peptides such as cyclopeptides containing portions of an epitope or variants thereof, where for example, one or several amino acids have been substituted or chemically modified in order to improve affinity to an antibody (mimotopes), can be used for constructing SADCs. A peptide screen can be performed with the aim of identifying peptides with optimized affinity to a disease-inducing autoantibody. The flexibility of structural or chemical peptide modification provided a solution to minimize the risk of immunogenicity, in particular of binding of the peptide to HLA and thus the risk of unwanted immune stimulation.

Therefore, wild-type as well as modified linear and circular peptide sequences were derived from a known epitope associated with an autoimmune disease. Peptides of various length and positions were systematically permutated by amino acid substitutions and synthesized on a peptide array. This allowed screening of 60000 circular and linear wild-type and mimotope peptides derived from these sequences. The peptide arrays were incubated with an autoantibody known to be involved in the autoimmune disease. This autoantibody was therefore used to screen the 60000 peptides and 100 circular and 100 linear peptide hits were selected based on their relative binding strength to the autoantibody. Of these 200 peptides, 51 sequences were identical between the circular and the linear peptide group. All of the best peptides identified had at least one amino acid substitution when aligned to the original sequences, respectively and are therefore regarded as mimotopes. It also turned out that higher binding strengths can be achieved with circularized peptides.

These newly identified peptides, preferentially those with high relative binding values, are used to generate SADCs that are able to remove autoantibodies directed against this particular epitope or to develop further mimotopes and derivatives based on their sequences.

### Example 5: Rapid, Selective Antibody Depletion in Mice using Various SADC Biopolymer Scaffolds

10 μg of model undesired antibody mAB anti V5 (Thermo Scientific) was injected i.p. into female Balb/c mice (5 animals per treatment group; aged 9-11 weeks) followed by intravenous injection of 50 μg SADC (different biopolymer scaffolds with tagged V5 peptides bound, see below) 48 hrs after the initial antibody administration. Blood was collected at 24 hrs intervals from the submandibular vein. Blood samples for time point 0 hrs were taken just before SADC administration.

Blood was collected every 24 hrs until time point 120 hrs after the SADC administration (x-axis). The decay and reduction of plasma anti-V5 IgG levels after SADC administration was determined by anti V5 titer readout using standard ELISA procedures in combination with coated V5-peptide-BSA (peptide sequence IPNPLLGLDC - SEQ ID NO: 134) and detection by goat anti mouse IgG bio (Southern Biotech, diluted 1:2000) as shown in FIG. 4. In addition, SADC levels (see Example 6) and immunocomplex formation (see Example 7) were analyzed.

EC50[OD450] values were determined using 4 parameter logistic curve fitting and relative signal decay between the initial level (set to 1 at time point 0) and the following time points (x-axis) was calculated as ratio of the EC50 values (y-axis, fold signal reduction EC50). All SADC peptides contained tags for direct detection of SADC and immunocomplexes from plasma samples; peptide sequences used for SADCs were: IPNPLLGLDGGSGDYKDDDDKGK(SEQ ID NO: 135)-(BiotinAca)GC (SADC with albumin scaffold-SADC-ALB, SADC with immunoglobulin scaffold -SADC-IG, SADC with haptoglobin scaffold - SADC-HP, and SADC with transferrin scaffold-SADC-TF) and unrelated peptide VKKIHIPSEKGGSGDYKDDDDKGK(SEQ ID NO: 136)-(BiotinAca)GC as negative control SADC (SADC-CTR).

The SADC scaffolds for the different treatment groups of 5 animals are displayed in black/grey shades (see inset of FIG. 4).

Treated groups exhibited rapid and pronounced antibody reduction already at 24 hrs (in particular SADC-TF) when compared to the mock treated control group SADC-CTL. SADC-CTR was used as reference for a normal antibody decay since it has no antibody lowering activity because its peptide sequence is not recognized by the administered anti V5 antibody. The decay of SADC-CTR is thus marked with a trend line, emphasizing the antibody level differences between treated and mock treated animals.

In order to determine the effectivity of selective antibody lowering under these experimental conditions, a two-way ANOVA test was performed using a Dunnett's multiple comparison test. 48 hrs after SADC administration, the antibody EC50 was highly significantly reduced in all SADC groups (p<0.0001) compared to the SADC-CTR reference group (trend line). At 120 hrs after SADC administration, antibody decrease was highly significant in the SADC-ALB and SADC-TF groups (both p<0.0001) and significant in the SADC-HP group (p=0.0292), whereas the SADC-IG group showed a trend towards an EC50 reduction(p=0.0722) 120 hrs after SADC administration. Of note, selective antibody reduction was highly significant (p<0.0001) in the SADC-ALB and SADC-TF groups at all tested time-points after SADC administration.

It is concluded that all SADC biopolymer scaffolds were able to selectively reduce antibody levels. Titer reduction was most pronounced with SADC-ALB and SADC-TF and no rebound or recycling of antibody levels was detected towards the last time points suggesting that undesired antibodies are degraded as intended.

### Example 6: Detection of SADCs in Plasma 24 hrs after SADC Injection

Plasma levels of different SADC variants at 24 hrs after i.v. injection into Balb/c mice. Determination of Plasma levels (y-axis) of SADC-ALB, -IG, -HP, -TF and the negative control SADC-CTR (x-axis), were detected in the plasmas from the animals already described in example 5. Injected plasma SADC levels were detected by standard ELISA whereby SADCs were captured via their biotin moieties of their peptides in combination with streptavidin coated plates (Thermo Scientific). Captured SADCs were detected by mouse anti Flag-HRP antibody (Thermo Scientific, 1:2,000 diluted) detecting the Flag-tagged peptides (see also example 7):

Assuming a theoretical amount in the order of 25 μg/ml in blood after injecting 50 μg SADC i.v., the detectable amount of SADC ranged between 799 and 623 ng/ml for SADC-ALB or SADC-IG and up to approximately 5000 ng/ml for SADC-TF, 24 hrs after SADC injection. However surprisingly and in contrast, SADC-HP and control SADC-CTR (which is also a SADC-HP variant, however carrying the in this case unrelated negative control peptide E006, see previous examples), had completely disappeared from circulation 24 hrs after injection, and were not detectable anymore. See FIG. 5.

This demonstrates that both Haptoglobin scaffold-based SADCs tested in the present example ((namely SADC-HP and SADC-CTR) exhibit a relatively shorter plasma half-life which represents an advantage over SADCs such as SADC-ALB, SADC-IG oder SADC-TF in regard of their potential role in complement-dependent vascular and renal damage due to the in vivo risk of immunocomplex formation. Another advantage of SADC-HP is the accelerated clearance rate of their unwanted target antibody from blood in cases where a rapid therapeutic effect is needed. The present results demonstrate that Haptoglobin-based SADC scaffolds (as represented by SADC-HP and SADC-CTR) are subject to rapid clearance from the blood, regardless of whether SADC-binding antibodies are present in the blood, thereby minimizing undesirable immunocomplex formation and showing rapid and efficient clearance. Haptoglobin-based SADCs such as SADC-HP in the present example thus provide a therapeutically relevant advantage over other SADC biopolymer scaffolds, such as demonstrated by SADC-TF or SADC-ALB, both of which are still detectable 24 hrs after injection under the described conditions, in contrast to SADC-HP or SADC-CTR which both are completely cleared 24 hrs after injection.

### Example 7: Detection of SADC-IgG Complexes in Plasma 24 hrs after SADC Injection

In order to determine the amount IgG bound to SADCs in vivo, after i.v. injection of 10 μg anti V5 IgG (Thermo Scientific) followed by injection of SADC-ALB, -HP, -TF and -CTR (50 μg) administered i.v. 48 h after antibody injection, plasma was collected from the submandibular vein, 24 hrs after SADC injection, and incubated on streptavidin plates for capturing SADCs from plasma via their biotinylated SADC-V5-peptide [IPNPLLGLDGGSGDYKDDDDKGK(SEQ ID NO: 135) (BiotinAca)GC or in case of SADC-CTR the negative control peptide VKKIHIPSEKGGSGDYKDDDDKGK(SEQ ID NO: 136) (BiotinAca)GC]. IgG bound to the streptavidin-captured SADCs was detected by ELISA using a goat anti mouse IgG HRP antibody (Jackson Immuno Research, diluted 1:2,000) for detection of the SADC-antibody complexes present in plasma 24 hrs after SADC injection. OD450 nm values (y-axis) obtained for a negative control serum from untreated animals were subtracted from the OD450nm values of the test groups (x-axis) for background correction.

As shown in FIG. 6, pronounced anti-V5 antibody signals were seen in case of SADC-ALB and SADC-TF injected mice (black bars represent background corrected OD values at a dilution of 1:25 , mean value of 5 mice; standard deviation error bars), whereas no antibody signal could be detected in plasmas from SADC-HP or control SADC-CTR injected animals (SADC-CTR is a negative control carrying the irrelevant peptide bio-FLG-E006 [VKKIHIPSEKGGSGDYKDDDDKGK(SEQ ID NO: 136)(BiotinAca)GC] that is not recognized by any anti V5 antibody). This demonstrates the absence of detectable amounts of SADC-HP/IgG complexes in the plasma 24 hrs after i.v. SADC application.

SADC-HP is therefore subject to accelerated clearance in anti V5 pre-injected mice when compared to SADC-ALB or SADC-TF.

### Example 8: In vitro Analysis of SADC-Immunoglobulin Complex Formation

SADC-antibody complex formation was analyzed by pre-incubating 1 μg/ml of human anti V5 antibody (anti V5 epitope tag [SVS-P-K], human IgG3, Absolute Antibody) with increasing concentrations of SADC-ALB, -IG, -HP, -TF and -CTR (displayed on the x-axis) in PBS+0.1% w/v BSA+0.1% v/v Tween20 for 2 hours at room temperature in order to allow for immunocomplex formation in vitro. After complex formation, samples were incubated on ELISA plates that had previously been coated with 10 μg/ml of human Clq (CompTech) for 1 h at room temperature, in order to allow capturing of in vitro formed immunocomplexes. Complexes were subsequently detected by ELISA using anti human IgG (Fab specific)-Peroxidase (Sigma, diluted 1:1,000). Measured signals at OD450 nm (y-axis) reflect Antibody-SADC complex formation in vitro.

As shown in FIG. 7, SADC-TF and -ALB showed pronounced immunocomplex formation and binding to Clq as reflected by the strong signals and by sharp signal lowering in case 1000ng/m1 SADC-TF due to the transition from antigen-antibody equilibrium to antigen excess. In contrast, in vitro immunocomplex formation with SADC-HP or SADC-IG were much less efficient when measured in the present assay.

Together with the in vivo data (previous examples), these findings corroborate the finding that haptoglobin scaffolds are advantageous over other SADC biopolymer scaffolds because of the reduced propensity to activate the complement system. In contrast, SADC-TF or SADC-ALB show higher complexation, and thereby carry a certain risk of activating the C1 complex with initiation of the classical complement pathway (a risk which may be tolerable in some settings, however).

### Example 9: Determination of IgG capturing by SADCs in vitro

Immunocomplexes were allowed to form in vitro, similar to the previous example, using 1 μg/ml mouse anti V5 antibody (Thermo Scientific) in combination with increasing amounts of SADCs (displayed on the x-axis). SADC-antibody complexes were captured on a streptavidin coated ELISA plate via the biotinylated SADC-peptides (see previous examples), followed by detection of bound anti-V5 using anti mouse IgG-HRP (Jackson Immuno Research, diluted 1:2,000).

Under these assay conditions, SADC-HP showed markedly less antibody binding capacity in vitro when compared to SADC-TF or

SADC-ALB (see FIG. 8, A). The calculated EC50 values for IgG detection on SADCs were 7.0 ng/ml, 27.9 ng/ml and 55.5 ng/ml for SADC-TF, -ALB and -HP, respectively (see FIG. 8, B).

This in vitro finding is consistent with the observation (see previous examples) that SADC-HP has a lower immunocomplex formation capacity when compared to SADC-TF or SADC-ALB which is regarded as a safety advantage with respect to its therapeutic use for the depletion of unwanted antibodies.

### Example 10: In-vivo Function of anti-CD163-Antibody-Based SADC Biopolymer Scaffold

Rapid in vivo blood clearance of anti-mouse-CD163 mAB E10B10 (as disclosed in WO 2011/039510 A2). mAB E10B10 was resynthesized with a mouse IgG2a backbone. 50 μg mAb E10B10 and Mac2-158 (human-specific anti-CD163 mAb as disclosed in WO 2011/039510 A2, used as negative control in this example since it does not bind to mouse CD163) were injected i.v. into mice and measured after 12, 24, 36, 48 , 72, 96 hours in an ELISA to determine the blood clearance.

mAb E10B10 was much more rapidly cleared from circulation than control mAb Mac2-158 was, as shown in FIG. 9, since E10B10 binds to the mouse CD163 whereas Mac2-158 is human-specific, although both were expressed as mouse IgG2a isotypes for direct comparison.

In conclusion, anti-CD163 antibodies are highly suitable as SADC scaffold because of their clearance profile. SADCs with such scaffolds will rapidly clear undesirable antibodies from circulation.

Detailed methods: 50 ug of biotinylated monoclonal antibodies E10B10 and biotinylated Mac2-158 were injected i.v. into mice and measured after 12, 24, 36, 48, 72, 96 hours to determine the clearance by ELISA: Streptavidin plates were incubated with plasma samples diluted in PBS+0.1% BSA+0.1% Tween20 for 1 h at room temperature (50 μl/well). After washing (3× with PBS+0.1% Tween20), bound biotinylated antibodies were detected with anti-mouse IgG+IgM-HRP antibody at a 1:1000 dilution. After washing, TMB substrate was added and development of the substrate was stopped with TMB Stop Solution. The signal at OD450 nm was read. The EC50 values were calculated by non-linear regression using 4 parametric curve fitting with constrained curves and least squares regression. EC50 values at time-point T12 (this was the first measured time-point after antibody injection) was set at 100%, all other EC50 values were compared to the levels at T12.

### Example 11: Identification of Peptides Binding to Factor VIII-Neutralizing Antibodies

In order to deplete, sequester or inactivate neutralizing antibodies against human factor VIII, prior to factor VIII administration, short, non-immunogenic peptides were searched, that could bind to the paratope of anti factor VIII antibodies.

The aim was to identify peptides that are recognized by anti factor VIII neutralizing antibodies. These peptides can then be attached to a scaffold of an SADC.

mAb BO2C11 represents a prototype neutralizing antibody that was isolated from hemophilic patients that had developed neutralizing antibodies (Jacquemin 1998). The antibody was recloned as a human/mouse chimeric antibody with mouse IgG2a constant chains and used for fine epitope mapping by peptide arrays.

Fine epitope mapping of monoclonal antibody BO2C11: BO2C11 represents a prototype neutralizing antibody that was generated upon isolation of PBMCs from a hemophila A patient with inhibitor. Cell lines were generated by immortalization with Epstein-Barr virus (EBV). The generated cell lines were tested for binding of antibody against the C2 domain of FVIII. In the course of this screening, the BO2C11 cell line was chosen because it secreted antibody binding to the C2 domain of FVIII and also inhibiting the FVIII activity. (Jacquemin, 1998). The antibody was subjected to fine epitope mapping using cyclic peptides derived from the factor VIII C2 domain (Genbank AAA52484.1) using cyclic peptide arrays.

The sequence at amino acid positions 2173 to 2351 of the factor VIII sequence, subunit C2 was used as a starting sequence for designing 7mer, lOmer and 13mer peptides. These peptides were synthesized and circularized on a peptide microarray and subsequently incubated with various concentrations of prototype neutralizing antibody BO2C11. The binding signal of mAB BO2C11 to the peptides yielded several factor VIII epitopes, designated epitope 1 (18 mer) and epitope 2 (16 mer).

Table 1 shows the alignments of the corresponding cyclic peptides that are bound by mAB BO2C11. The number of the peptide designations corresponds to the ranked binding signal of the antibody to the peptide microarray (i.e. peptide 01 binds strongest, 02 second strongest, etc.) out of up to 50 top binders that were selected for alignment to the factor VIII sequence using Clustal Omega (https://www.ebi.ac.uk/Tools/msa/clustalo/).

Fine epitope mapping of monoclonal antibody GMA-8015: GMA-8015 represents a prototype neutralizing antibody that was generated by conventional mouse hybridoma technique by immunizing hemophila A mice with Factor VIII (Healey et al, 2007). It has been shown to neutralize Factor VIII as assessed by the Bethesda assay. In addition, antibody GMA-8015 (also known as antibody 4A4) is used to induce acquired hemophilia in mice (Keshava et al, 2017).

GMA-8015 was subjected to fine epitope mapping using cyclic peptides derived from the factor VIII A2 domain (Genbank AAA52484.1) using cyclic peptide arrays, as for mAB BO2C11. The diversity of the immune response to the A2 domain has previously been described e.g. by Markovitz et al, 2013. The sequence at amino acid positions 337 to 710 of the factor VIII sequence, subunit A2 was used as a starting sequence for designing 7mer, lOmer and 13mer peptides were synthesized and circularized on a peptide microarray and subsequently incubated with various concentrations of prototype neutralizing antibody GMA-8015. The binding signal of GMA-8015 to the peptides yielded several factor VIII epitopes, designated epitope 3 (20 mer) and epitope 4 (14 mer). Table 1 shows the alignments of the corresponding cyclic peptides that are bound by mAB GMA-8015. The number of the peptide designations corresponds to the ranked binding signal of the antibody to the peptide microarray (i.e. peptide 01 binds strongest, 02 second strongest, etc.) out of up to 50 top binders that were selected for alignment to the factor VIII sequence.

Fine epitope mapping of monoclonal antibody GMA-8021: GMA-8021 represents a prototype neutralizing antibody that was generated by conventional mouse hybridoma technique by immunizing hemophila A mice with Factor VIII (Healey et al, 2007). It has been shown to neutralize Factor VIII as assessed by the Bethesda assay. The antibody was subjected to fine epitope mapping using cyclic peptides derived from the factor VIII A2 domain (Genbank AAA52484.1) using cyclic peptide arrays, as for mAB BO2C11.

The sequence at amino acid positions 337 to 710 of the factor VIII sequence, subunit A2 was used as a starting sequence for designing 7mer, lOmer and 13mer peptides were synthesized and circularized on a peptide microarray and subsequently incubated with various concentrations of prototype neutralizing antibody GMA-8021. The binding signal of GMA-8021 to the peptides yielded several factor VIII epitopes, designated epitope 5 (16 mer), epitope 6 (15 mer), and epitope 7 (16 mer). Table 1 shows the alignments of the corresponding cyclic peptides that are bound by mAB GMA-8021. The number of the peptide designations corresponds to the ranked binding signal of the antibody to the peptide microarray (i.e. peptide 01 binds strongest, 02 second strongest, etc.) out of up to 50 top binders that were selected for alignment to the factor VIII sequence.

Fine epitope mapping of monoclonal antibody GMA-8014: GMA-8014 represents a prototype neutralizing antibody that was generated by conventional mouse hybridoma technique by immunizing hemophila A mice with Factor VIII (Healey et al, 2007). It has been shown to neutralize Factor VIII as assessed by the Bethesda assay. The antibody was subjected to fine epitope mapping using cyclic peptides derived from the factor VIII C2 domain (Genbank AAA52484.1) using cyclic peptide arrays, as for mAB BO2C11.

The sequence at amino acid positions 2173 to 2351 of the factor VIII sequence, subunit C2 was used as a starting sequence for designing 7mer, lOmer and 13mer peptides were synthesized and circularized on a peptide microarray and subsequently incubated with various concentrations of prototype neutralizing antibody GMA-8014. The binding signal of GMA-8014 to the peptides yielded several factor VIII epitopes, designated epitope 8 (10 mer), epitope 9 (15 mer), epitope 10 (10 mer), epitope 11 (15 mer) and epitope 12 (13 mer). Table 1 shows the alignments of the corresponding cyclic peptides that are bound by mAB GMA-8014. The number of the peptide designations corresponds to the ranked binding signal of the antibody to the peptide microarray (i.e. peptide 01 binds strongest, 02 second strongest, etc.) out of up to 50 top binders that were selected for alignment to the factor VIII sequence.

Fine epitope mapping of monoclonal antibody GMA-8008: GMA-8008 represents a prototype neutralizing antibody that was generated by conventional mouse hybridoma technique by immunizing hemophila A mice with Factor VIII (Healey et al, 2007). It has been shown to neutralize Factor VIII as assessed by the Bethesda assay. The antibody was subjected to fine epitope mapping using cyclic peptides derived from the factor VIII C2 domain (Genbank AAA52484.1) using cyclic peptide arrays, as for mAB BO2C11.

The sequence at amino acid positions 2173 to 2351 of the factor VIII sequence, subunit C2 was used as a starting sequence for designing 7mer, lOmer and 13mer peptides were synthesized and circularized on a peptide microarray and subsequently incubated with various concentrations of prototype neutralizing antibody GMA-8008. The binding signal of GMA-8008 to the peptides yielded several factor VIII epitopes, designated epitope 13 (13 mer), epitope 14 (9 mer) and epitope 15 (19 mer). Table 1 shows the alignments of the corresponding cyclic peptides that are bound by mAB GMA-8008. The number of the peptide designations corresponds to the ranked binding signal of the antibody to the peptide microarray (i.e. peptide 01 binds strongest, 02 second strongest, etc.) out of up to 50 top binders that were selected for alignment to the factor VIII sequence.

Taken together, the identified epitope sequences present on neutralizing prototypic antibodies were:

BO2C11:

epitope1:
(SEQ ID NO: 1)
STLRMELMGCDLNSCSMP (aa 2179-2196 based
on Genbank AAA52484.1)
epitope2:
(SEQ ID NO: 2)
IALRMEVLGCEAQDLY (aa 2336-2351 based on
Genbank AAA52484.1)
GMA-8015
epitope3:
(SEQ ID NO: 3)
QYLNNGPQRIGRKYKKVRFM (aa 429-448 based
on Genbank AAA52484.1)
epitope4:
(SEQ ID NO: 4)
LYGEVGDTLLIIFK (aa 472-485 based on
Genbank AAA52484.1)
GMA-8021
epitope05:
(SEQ ID NO: 5)
NGPQRIGRKYKKVRFM (aa 433-448 based on
Genbank AAA52484.1)
epitope06:
(SEQ ID NO: 6)
KSQYLNNGPQRIGRK (aa 427-441 based on
Genbank AAA52484.1)
epitope07:
(SEQ ID NO: 7)
PHGITDVRPLYSRRLP (aa 496-511 based on
Genbank AAA52484.1)
GMA-8014
epitope08:
(SEQ ID NO: 8)
THYSIRSTLR (aa 2173-2182 based on
Genbank AAA52484.1)
epitope09:
(SEQ ID NO: 9)
KARLHLQGRSNAWRP (aa 2226-2240 based on
Genbank AAA52484.1)
epitope10:
(SEQ ID NO: 10)
QDGHQWTLFF (aa 2285-2294 based on
Genbank AAA52484.1)
epitope11:
(SEQ ID NO: 11)
NSLDPPLLTRYLRIH (aa 2314-2328 based on
Genbank AAA52484.1)
epitope12:
(SEQ ID NO: 12)
IHPQSWVHQIALR (aa 2327-2339 based on
Genbank AAA52484.1)
GMA-8008
epitope13:
(SEQ ID NO: 13)
SSSQDGHQWTLFF (aa 2282-2294 based on
Genbank AAA52484.1)
epitope14:
(SEQ ID NO: 14)
MGCDLNSCS (aa 2186-2194 based on
Genbank AAA52484.1)
epitope15:
(SEQ ID NO: 15)
VHQIALRMEVLGCEAQDLY (aa 2333-2351
based on Genbank AAA52484.1)

Remarkably, it was found that the following epitopes are shared between pairs of antibodies and that they overlap in two out of three cases pointing to general structural accessibility of Factor VIII epitopes by anti Factor VIII antibodies:

epitope10
(SEQ ID NO: 10)
---QDGHQWTLFF
epitope13
(SEQ ID NO: 13)
SSSQDGHQWTLFF
epitope02
(SEQ ID NO: 2)
---IALRMEVLGCEAQDLY
epitope15
(SEQ ID NO: 15)
VHQIALRMEVLGCEAQDLY
epitope06
(SEQ ID NO: 6)
KSQYLNNGPQRIGRK-------
epitope03
(SEQ ID NO: 3)
--QYLNNGPQRIGRKYKKVRFM

Based on epitope06 and epitope03, a longer epitopel6 can be defined: KSQYLNNGPQRIGRKYKKVRFM (SEQ ID NO: 16).

From these epitopes, peptides can be designed that are recognized by neutralizing anti FVIII antibodies. These peptides can be used for the construction of SADCs, for the development of new mimotopes, and for the development of peptides for neutralizing antibody diagnostics and -typing in patients with hemophilia.

TABLE 1

| SEQ ID NO: | PEPTIDE | SEQUENCE |
|---|---|---|
| 1 | epitope01 | STLRMELMGCDLNSCSMP |
| 17 | 02 | -----ELMGCDLNSC--- |
| 18 | 03 | --------GCDLNSC--- |
| 19 | 06 | -----ELMGCDL------ |
| 20 | 07 | ---------CDLNSCS-- |
| 21 | 08 | ----MELMGCD------- |
| 22 | 10 | STLRMELMGC-------- |
| 23 | 11 | --------GCDLNSCSMP |
| 24 | 15 | -----------LNSCSMP |
| 25 | 17 | ---RMELMGCDLN----- |
| 26 | 18 | ---RMELMGC-------- |
| 27 | 20 | --LRMELMGCDLNSC--- |
| 28 | 24 | ------LMGCDLN----- |
| 29 | 30 | -----ELMGCDLNSCSMP |
| 2 | epitope02 | IALRMEVLGCEAQDLY-- |
| 30 | 01 | ---------CEAQDLY-- |
| 31 | 04 | ------VLGCEAQDLY-- |
| 32 | 05 | ---RMEVLGCEAQDLY-- |
| 33 | 09 | ----MEVLGCE------- |
| 34 | 12 | ----MEVLGCEAQDLYG- |
| 35 | 13 | -----EVLGCEAQDLYGS |
| 36 | 14 | ---RMEVLGC-------- |
| 37 | 16 | -----EVLGCEA------ |
| 38 | 19 | IALRMEVLGC-------- |
| 3 | epitope3 | QYLNNGPQRIGRKYKKVRFM |
| 39 | 01 | -----GPQRIGRKYKKVR-- |
| 40 | 02 | ------PQRIGRKYKKVRF- |
| 41 | 03 | --------RIGRKYKKVR-- |
| 42 | 04 | ---NNGPQRIGRKYKK---- |
| 43 | 05 | -------QRIGRKYKKV--- |
| 44 | 06 | ----NGPQRIGRKYKKV--- |
| 45 | 07 | -------QRIGRKYKKVRFM |
| 46 | 08 | ------PQRIGRKYKK---- |
| 47 | 10 | --LNNGPQRIGRKYK----- |
| 48 | 11 | ---------IGRKYKKVRF- |
| 49 | 18 | -----GPQRIGRKYK----- |
| 50 | 20 | -----------RKYKKVR-- |
| 51 | 21 | QYLNNGPQRIGRK------- |
| 4 | epitope4 | LYGEVGDTLLIIFK |
| 52 | 09 | ---EVGDTLLIIF- |
| 53 | 12 | LYGEVGDTLLIIF- |
| 54 | 13 | ------DTLLIIF- |
| 55 | 15 | ----VGDTLLIIFK |
| 56 | 17 | --GEVGDTLLII-- |
| 57 | 19 | -------TLLIIFK |
| 5 | epitope05 | NGPQRIGRKYKKVRFM |
| 58 | 02 | -GPQRIGRKYKKVR-- |
| 59 | 04 | NGPQRIGRKYKKV--- |
| 60 | 06 | --PQRIGRKYKKVRF- |
| 61 | 07 | ----RIGRKYKKVR-- |
| 62 | 09 | ---QRIGRKYKKV--- |
| 63 | 16 | ---QRIGRKYKKVRFM |
| 64 | 18 | -------RKYKKVR-- |
| 65 | 20 | --PQRIGRKYKK---- |
| 66 | 21 | -GPQRIGR-------- |
| 67 | 25 | -GPQRIGRKYK----- |
| 68 | 26 | --PQRIGRK------- |
| 6 | epitope06 | KSQYLNNGPQRIGRK |
| 69 | 13 | KSQYLNNGPQRIG-- |
| 70 | 14 | -SQYLNNGPQRIGR- |
| 71 | 22 | --QYLNNGPQRIGRK |
| 72 | 23 | ----LNNGPQRIGR- |
| 73 | 28 | ---YLNNGPQRIG-- |
| 74 | 29 | -----NNGPQRIGRK |
| 75 | 30 | ------DGPTKSD-- |
| 7 | epitope07 | PHGITDVRPLYSRRLP |
| 76 | 01 | -HGITDVRPLYSRR-- |
| 77 | 03 | PHGITDVRPLYSR--- |
| 78 | 05 | ---ITDVRPLYSRRLP |
| 79 | 08 | --GITDVRPLYSRRL- |
| 80 | 10 | ----TDVRPLYSRR-- |
| 81 | 11 | -------RPLYSRR-- |
| 82 | 12 | ---ITDVRPLYSR--- |
| 83 | 15 | -----DVRPLYSRRL- |
| 84 | 19 | ------VRPLYSRRLP |
| 85 | 24 | ------VRPLYSR--- |
| 86 | 30 | --------PRCLTR-- |
| 8 | epitope08 | ---THYSIRSTLR |
| 87 | 02 | ---THYSIRSTLR |
| 88 | 03 | GSGTHYSIRSTLR |
| 9 | epitope09 | KARLHLQGRSNAWRP |
| 89 | 01 | -ARLHLQGRSNAWR- |
| 90 | 04 | ----HLQGRSNAWR- |
| 91 | 06 | KARLHLQGRSNAW-- |
| 92 | 09 | --RLHLQGRSNAWRP |
| 93 | 14 | ---LHLQGRSNAW-- |
| 94 | 19 | -------GRSNAWR- |
| 10 | epitope10 | QDGHQWTLFF |
| 95 | 8 | QDGHQWTLFF |
| 96 | 11 | --GHQWTLF- |
| 97 | 20 | ---HQWTLFF |
| 11 | epitope11 | NSLDPPLLTRYLRIH |
| 98 | 05 | ------LLTRYLR-- |
| 99 | 07 | -------LTRYLRI- |
| 100 | 10 | -----PLLTRYL--- |
| 101 | 13 | --------TRYLRIH |
| 102 | 15 | NSLDPPLLTRYLR-- |
| 103 | 16 | ---DPPLLTRYLR-- |
| 104 | 17 | ----PPLLTRYLRI- |
| 12 | epitope12 | IHPQSWVHQIALR |
| 105 | 12 | IHPQSWVHQIALR |
| 106 | 18 | ------VHQIALR |
| 13 | epitope13 | SSSQDGHQWTLFF |
| 107 | 01 | ------HQWTLFF |
| 108 | 03 | ---QDGHQWTLFF |
| 109 | 13 | SSSQDGHQWTLFF |
| 110 | 15 | --SQDGHQWTLF- |
| 111 | 24 | --SQDGHQW---- |
| 112 | 31 | -----GHQWTLF- |
| 14 | epitope14 | -------MGCDLNSCS |
| 113 | 02 | --------GCDLNSC- |
| 114 | 05 | --LRMELMGCDL---- |
| 115 | 07 | -----ELMGCDLNSC- |
| 116 | 08 | STLRMELMGC------ |
| 117 | 17 | ------LMGCDLNSCS |
| 118 | 18 | -----ELMGCDL---- |
| 119 | 19 | -TLRMELMGCD----- |
| 120 | 21 | --LRMELMGCDLNSC- |
| 15 | epitope15 | VHQIALRMEVLGCEAQDLY- |
| 121 | 09 | ------RMEVLGC------- |
| 122 | 12 | ---IALRMEVLGC------- |
| 123 | 22 | ----ALRMEVLGCE------ |
| 124 | 23 | VHQIALRMEVLGC------- |
| 125 | 34 | ----------LGCEAQDLYG |
| 126 | 35 | --------EVLGCEA----- |
| 16 | epitope16 | KSQYLNNGPQRIGRKYKKVRFM |

### Example 12: Administration of SADCs to Hemophilia A Patients

SADCs are prepared essentially as described in Example 1, using human transferrin as biopolymer scaffold.

N-terminally cysteinylated peptide SEQ ID NO. 39 and/or C-terminally cysteinylated peptide SEQ ID NO. 107 are linked to the scaffold using sulfo-GMBS-activated human transferrin, thereby providing transferrin-based SADCs with the corresponding cysteinylated peptides, that are thereby covalently attached to the lysines of the corresponding biopolymer scaffold. These SADC conjugates are purified and resuspended in PBS.

To three hemophilia A patients undergoing treatment with human factor VIII and having developed neutralizing antibodies against human factor VIII, 150 mg, 250 mg, and 500 mg, respectively, of resuspended SADC conjugate is administered intravenously, in order to reduce neutralizing antibodies in the plasma of the patients and thereby increasing the efficacy of the treatment with human factor VIII.

### NON-PATENT REFERENCES

Ananyeva, Natalya M., et al. “Inhibitors in hemophilia A: mechanisms of inhibition, management and perspectives.” Blood coagulation & fibrinolysis 15.2 (2004): 109-124.
Armstrong, Jonathan K. “The occurrence, induction, specificity and potential effect of antibodies against poly (ethylene glycol).” Pegylated protein drugs: Basic science and clinical applications. Birkhäuser Basel, 2009. 147-168.
Carter, John Mark, and Larry Loomis-Price. “B cell epitope mapping using synthetic peptides.” Current protocols in immunology 60.1 (2004): 9-4.
Dijkstra, C. D., et al. “The heterogeneity of mononuclear phagocytes in lymphoid organs: distinct macrophage subpopulations in rat recognized by monoclonal antibodies ED1, ED2 and ED3.” Microenvironments in the Lymphoid System. Springer, Boston, MA, 1985. 409-419.
Elliott, Serra E., et al. “A pre-eclampsia-associated Epstein-Barr virus antibody cross-reacts with placental GPR50.” Clinical Immunology 168 (2016): 64-71.
Erlandsson, Ann, et al. “In vivo clearing of idiotypic antibodies with antiidiotypic antibodies and their derivatives.” Molecular immunology 43.6 (2006): 599-606.
Etzerodt, Anders, et al. “Efficient intracellular drug-targeting of macrophages using stealth liposomes directed to the hemoglobin scavenger receptor CD163.” Journal of controlled release 160.1 (2012): 72-80.
Fabriek, Babs O., et al. “The macrophage scavenger receptor CD163 functions as an innate immune sensor for bacteria.” Blood 113.4 (2009): 887-892.
Franchini, Massimo, et al. “Acquired hemophilia A: a review of recent data and new therapeutic options.” Hematology 22.9 (2017): 514-520.
Garces, Jorge Carlos, et al. “Antibody-mediated rejection: a review.” The Ochsner Journal 17.1 (2017): 46.
Gazarian, Karlen, et al. “Mimotope peptides selected from phage display combinatorial library by serum antibodies of pigs experimentally infected with Taenia solium as leads to developing diagnostic antigens for human neurocysticercosis.” Peptides 38.2 (2012): 381-388.
Gfeller, David, et al. “Current tools for predicting cancer-specific T cell immunity.” Oncoimmunology 5.7 (2016): e1177691.
Giangrande, Paul L F, et al. “European principles of inhibitor management in patients with haemophilia.” Orphanet journal of rare diseases 13.1 (2018): 1-6.
Granfeldt, Asger, et al. “Targeting dexamethasone to macrophages in a porcine endotoxemic model.” Critical Care Medicine 41.11 (2013): e309-e318.
Graversen, Jonas H., et al. “Targeting the hemoglobin scavenger receptor CD163 in macrophages highly increases the anti-inflammatory potency of dexamethasone.” Molecular Therapy 20.8 (2012): 1550-1558.
Gurda, Brittney L., et al. “Mapping a neutralizing epitope onto the capsid of adeno-associated virus serotype 8.” Journal of virology 86.15 (2012): 7739-7751.
Hansen, Lajla Bruntse, Soren Buus, and Claus Schafer-Nielsen. “Identification and mapping of linear antibody epitopes in human serum albumin using high-density peptide arrays.” PLoS One 8.7 (2013): e68902.
Healey, J. F., et al. “The humoral response to human factor VIII in hemophilia A mice.” Journal of Thrombosis and Haemostasis 5.3 (2007): 512-519.
Homma, Masayuki, et al. “A Novel Fusion Protein, AChR-Fc, Ameliorates Myasthenia Gravis by Neutralizing Antiacetylcholine Receptor Antibodies and Suppressing Acetylcholine Receptor-Reactive B Cells.” Neurotherapeutics 14.1 (2017): 191-198.
Howard Jr, James F. “Myasthenia gravis: the role of complement at the neuromuscular junction.” Annals of the New York Academy of Sciences 1412.1 (2018): 113-128.
Howarth, M., & Brune, K. D. (2018). New routes and opportunities for modular construction of particulate vaccines: stick, click and glue. Frontiers in immunology, 9, 1432.
Jacquemin, Marc G., et al. “Mechanism and kinetics of factor VIII inactivation: study with an IgG4 monoclonal antibody derived from a hemophilia A patient with inhibitor.” Blood, The Journal of the American Society of Hematology 92.2 (1998): 496-506.
Jansson, Liselotte, et al. “Immunotherapy With Apitopes Blocks the Immune Response to TSH Receptor in HLA-DR Transgenic Mice.” Endocrinology 159.9 (2018): 3446-3457.
Jensen, Kamilla Kjaergaard, et al. “Improved methods for predicting peptide binding affinity to MHC class II molecules.” Immunology 154.3 (2018): 394-406.
Jurtz, Vanessa, et al. “NetMHCpan-4.0: improved peptide-MHC class I interaction predictions integrating eluted ligand and peptide binding affinity data.” The Journal of Immunology 199.9 (2017): 3360-3368.
Kainulainen, Markus H., et al. “High-throughput quantitation of SARS-CoV-2 antibodies in a single-dilution homogeneous assay.” Scientific reports 11.1 (2021): 1-9.
Keshava, Shiva, et al. “Factor VIIa interaction with EPCR modulates the hemostatic effect of rFVIIa in hemophilia therapy: mode of its action.” Blood advances 1.15 (2017): 1206-1214.
Konkle, Barbara A et al. “Hemophilia A” In: GeneReviews° [Internet]. Seattle (WA): University of Washington, Seattle; 1993-2021. 2000 Sep 21 [updated 2017 Jun. 22].
Koşaloğlu-Yalçin, Zeynep, et al. “Predicting T cell recognition of MHC class I restricted neoepitopes.” Oncoimmunology 7.11 (2018): e1492508.
Lacroix-Desmazes, Sebastien, et al. “Tolerating factor VIII: recent progress.” Frontiers in immunology 10 (2020): 2991.
Lavigne-Lissalde, Géraldine, et al. “Anti-factor VIII antibodies.” Thrombosis and haemostasis 94.10 (2005): 760-769.
Lazaridis, Konstantinos, et al. “Specific removal of autoantibodies by extracorporeal immunoadsorption ameliorates experimental autoimmune myasthenia gravis.” Journal of neuroimmunology 312 (2017): 24-30.
Leung, Nicki YH, et al. “Screening and identification of mimotopes of the major shrimp allergen tropomyosin using one-bead-one-compound peptide libraries.” Cellular & molecular immunology 14.3 (2017): 308-318.
Li, Peipei, Li Wang, and Li-jun Di. “Applications of protein fragment complementation assays for analyzing biomolecular interactions and biochemical networks in living cells.” Journal of proteome research 18.8 (2019): 2987-2998.
Lim, Sung In, and Inchan Kwon. “Bioconjugation of therapeutic proteins and enzymes using the expanded set of genetically encoded amino acids.” Critical reviews in biotechnology 36.5 (2016): 803-815.
Lin, Chia-Hao, et al. “Identification of a major epitope by anti-interferon-y autoantibodies in patients with mycobacterial disease.” Nature medicine 22.9 (2016): 994.
Ljung, Rolf C R. “Intracranial haemorrhage in haemophilia A and B.” British journal of haematology 140.4 (2008): 378-384.
Lorentz, Kristen M., et al. “Engineered binding to erythrocytes induces immunological tolerance to E. coli asparaginase.” Science advances 1.6 (2015): e1500112.
Luo, Jie, et al. “Main immunogenic region structure promotes binding of conformation-dependent myasthenia gravis autoantibodies, nicotinic acetylcholine receptor conformation maturation, and agonist sensitivity.” Journal of Neuroscience 29.44 (2009): 13898-13908.
Luo, Jie, and Jon Lindstrom. “AChR-specific immunosuppressive therapy of myasthenia gravis.” Biochemical pharmacology 97.4 (2015): 609-619.
Madsen, Mette, et al. “Molecular Characterization of the Haptoglobin Hemoglobin Receptor CD163 ligand binding properties of the scavenger receptor cysteine-rich domain region.” Journal of Biological Chemistry 279.49 (2004): 51561-51567.
Majowicz, Anna, et al. “Seroprevalence of pre-existing NABS against AAV1, 2, 5, 6 and 8 in the South African Hemophilia B patient population.” (2019): 3353-3353.
Markovitz, Rebecca C., et al. “The diversity of the immune response to the A2 domain of human factor VIII.” Blood, The Journal of the American Society of Hematology 121.14 (2013): 2785-2795.
Mazor, Ronit, et al. “Tolerogenic nanoparticles restore the antitumor activity of recombinant immunotoxins by mitigating immunogenicity.” Proceedings of the National Academy of Sciences 115.4 (2018): E733-E742.
Meister, Daniel, S. Maryamdokht Taimoory, and John F. Trant. “Unnatural amino acids improve affinity and modulate immunogenicity: Developing peptides to treat MHC type II autoimmune disorders.” Peptide Science 111.1 (2019): e24058.
Mingozzi, Federico, et al. “Overcoming preexisting humoral immunity to AAV using capsid decoys.” Science translational medicine 5.194 (2013): 194ra92-194ra92.
Mingozzi, Federico, and Katherine A. High. “Overcoming the host immune response to adeno-associated virus gene delivery vectors: the race between clearance, tolerance, neutralization, and escape.” Annual review of virology 4 (2017): 511-534.
Morimoto et. al., Bioconjugate Chemistry 25 (8) (2014): 1479-1491
Moussa, Ehab M., et al. “Immunogenicity of therapeutic protein aggregates.” Journal of pharmaceutical sciences 105.2 (2016): 417-430.
Müller, Manuel M. “Post-translational modifications of protein backbones: unique functions, mechanisms, and challenges.” Biochemistry 57.2 (2017): 177-185.
O'Mahony, Brian. “Haemophilia care in Europe: Past progress and future promise.” Haemophilia 26.5 (2020): 752-758.
Siang Ong, Yong, et al. “Recent advances in synthesis and identification of cyclic peptides for bioapplications.” Current topics in medicinal chemistry 17.20 (2017): 2302-2318.
Patel, Seema R., et al. “The immune response to the fVIII gene therapy in preclinical models.” Frontiers in immunology 11 (2020): 494.
Peters, Bjoern, et al. “A community resource benchmarking predictions of peptide binding to MHC-I molecules.” PLoS computational biology 2.6 (2006): e65.
Peters, Rob, and Tim Harris. “Advances and innovations in haemophilia treatment.” Nature Reviews Drug Discovery 17.7 (2018): 493-508.
Pishesha, Novalia, et al. “Engineered erythrocytes covalently linked to antigenic peptides can protect against autoimmune disease.” Proceedings of the National Academy of Sciences (2017): 201701746.
Pratt, Kathleen P., Valder R. Arruda, and Sébastien Lacroix-Desmazes. “Inhibitors-Recent insights.” Haemophilia 27 (2021): 28-36.
Rey et al., Clinical Immunology 96 (3) (2000): 269-279
Rice et al., EMBOSS: the European Molecular Biology Open Software Suite, Trends Genet. 2000 Jun;16(6):276-7
Ruff, Robert L., and Robert P. Lisak. “Nature and action of antibodies in myasthenia gravis.” Neurologic clinics 36.2 (2018): 275-291.
Rummler, Silke, et al. “Current techniques for ABO-incompatible living donor liver transplantation.” World journal of transplantation 6.3 (2016): 548.
Runcie, Karie, et al. “Bi-specific and tri-specific antibodies-the next big thing in solid tumor therapeutics.” Molecular Medicine 24.1 (2018): 50.
Ryan, Brent J., Ahuva Nissim, and Paul G. Winyard. “Oxidative post-translational modifications and their involvement in the pathogenesis of autoimmune diseases.” Redox biology 2 (2014): 715-724.
Shanmugam, Arulkumaran, et al. “Identification of PSA peptide mimotopes using phage display peptide library.” Peptides 32.6 (2011): 1097-1102.
Skytthe, Maria K., Jonas Heilskov Graversen, and Soren K. Moestrup. “Targeting of CD163+Macrophages in Inflammatory and Malignant Diseases.” International Journal of Molecular Sciences 21.15 (2020): 5497.
Sorensen, Karen Kristine, et al. “Liver sinusoidal endothelial cells.” Comprehensive Physiology 5.4 (2011): 1751-1774.
Spiess, Christoph, Qianting Zhai, and Paul J. Carter. “Alternative molecular formats and therapeutic applications for bispecific antibodies.” Molecular immunology 67.2 (2015): 95-106.
Taddeo, Adriano, et al. “Selection and depletion of plasma cells based on the specificity of the secreted antibody.” European journal of immunology 45.1 (2015): 317-319.
Teschner, Sven, et al. “ABC-incompatible kidney transplantation using regenerative selective immunoglobulin adsorption.” Journal of clinical apheresis 27.2 (2012): 51-60.
Tetala, Kishore K R, et al. “Selective depletion of neuropathy-related antibodies from human serum by monolithic affinity columns containing ganglioside mimics.” Journal of medicinal chemistry 54.10 (2011): 3500-3505.
Villard, Sylvie, et al. “Peptide decoys selected by phage display block in vitro and in vivo activity of a human anti-FVIII inhibitor.” Blood 102.3 (2003): 949-952.
Vincent, Angela, et al. “Serological and experimental studies in different forms of myasthenia gravis.” Annals of the New York Academy of Sciences 1413.1 (2018): 143-153.
Wallukat, Gerd, et al. “Patients with preeclampsia develop agonistic autoantibodies against the angiotensin AT 1 receptor.” The Journal of clinical investigation 103.7 (1999): 945-952.
Zhou, Cissy C., et al. “Angiotensin receptor agonistic autoantibodies induce pre-eclampsia in pregnant mice.” Nature medicine 14.8 (2008): 855.

1. A compound comprising: a biopolymer scaffold and at least two peptides derived from factor VIII with a sequence length of 6-13 amino acids;
wherein each of the peptides independently comprises a 6-amino-acid fragment; and
wherein at most three amino acids are independently substituted by any other amino acid.

2. The compound of claim 1, wherein each of the peptides independently comprises a 6-amino-acid fragment of an amino-acid sequence selected from the group consisting of:
STLRMELMGCDLNSCSMP (SEQ ID NO: 1), IALRMEVLGCEAQDLY (SEQ ID NO: 2), QYLNNGPQRIGRKYKKVRFM (SEQ ID NO: 3), LYGEVGDTLLIIFK (SEQ ID NO: 4), NGPQRIGRKYKKVRFM (SEQ ID NO: 5), KSQYLNNGPQRIGRK (SEQ ID NO: 6), PHGITDVRPLYSRRLP (SEQ ID NO: 7), THYSIRSTLR (SEQ ID NO: 8), KARLHLQGRSNAWRP (SEQ ID NO: 9), QDGHQWTLFF (SEQ ID NO: 10), NSLDPPLLTRYLRIH (SEQ ID NO: 11), IHPQSWVHQIALR (SEQ ID NO: 12), SSSQDGHQWTLFF (SEQ ID NO: 13), MGCDLNSCS (SEQ ID NO: 14), VHQIALRMEVLGCEAQDLY (SEQ ID NO: 15), and KSQYLNNGPQRIGRKYKKVRFM (SEQ ID NO: 16),
wherein at most three amino acids are independently substituted by any other amino acid.

3. The compound of claim 1, wherein the at least two peptides comprise a peptide P₁ and a peptide P₂, wherein P₁ and P₂ independently comprise a 6-amino-acid fragment, as defined in claim 1, wherein P₁ and P₂ are present in form of a peptide dimer P₁ -S-P₂, wherein S is a non-peptide spacer, wherein the peptide dimer is covalently bound to the biopolymer scaffold, preferably via a linker.

4. The compound of claim 1, wherein the biopolymer scaffold is selected from human globulins and human albumin.

5. The compound of claim 1, wherein at least one of the at least two peptides are circularized.

6. The compound of claim 1, wherein the compound is non-immunogenic in humans.

7. The compound of claim 1, wherein each of the peptides independently comprises an amino-acid sequence selected from SEQ ID NOs: 17 to 126, optionally wherein at most three amino acids are independently substituted by any other amino acid, or a 6-, amino-acid fragment thereof.

8. The compound of claim 1, wherein each of the peptides independently consists of an amino-acid sequence selected from SEQ ID NOs: 17 to 126, wherein at most three amino acids are independently substituted by any other amino acid, optionally with an N-terminal and/or C-terminal cysteine residue.

9. The compound of claim 1, wherein the biopolymer scaffold is human transferrin.

10. A pharmaceutical composition comprising the compound of claim 1 and at least one pharmaceutically acceptable excipient.

11. The pharmaceutical composition of claim 10, wherein the molar ratio of the peptides to scaffold in the composition is from 2:1 to 100.1.

12. The pharmaceutical composition of claim 10, further comprising a factor VIII replacement product.

13. The pharmaceutical composition of claim 10, for use in prevention or treatment of hemophilia A in an individual.

14. The pharmaceutical composition for use according to claim 13, wherein the pharmaceutical composition is administered to the individual, and
wherein a factor VIII replacement product is administered to the individual in combination with said composition, before said composition is administered, or after said composition has been administered,
wherein said composition is administered at least twice within a 96-hour window, wherein the window is followed by administration of the factor VIII replacement product within 24 hours.

15. The pharmaceutical composition according to claim 10, for use in inhibiting neutralization and/or inhibition of a factor VIII replacement product in an individual, wherein the pharmaceutical composition is administered at least twice within a 96-hour window, wherein the window is followed by administration of the factor VIII replacement product within 24 hours.
